SEQUENCE LISTING (45 sequences)

SEQ ID NO: 1 - DNA, 19 bp, Artificial Sequence
  Synthetic
ccctggccgtgctagacaa

SEQ ID NO: 2 - DNA, 19 bp, Artificial Sequence
  Synthetic
ggcatggtgttactgtgta

SEQ ID NO: 3 - DNA, 19 bp, Artificial Sequence
  Synthetic
cgagctaaagctcaaattg

SEQ ID NO: 4 - DNA, 19 bp, Artificial Sequence
  Synthetic
ggctttatcctcctttgaa

SEQ ID NO: 5 - DNA, 19 bp, Artificial Sequence
  Synthetic
ccgagttcctcttatccta

SEQ ID NO: 6 - DNA, 19 bp, Artificial Sequence
  Synthetic
cagatgacctggagcctat

SEQ ID NO: 7 - DNA, 19 bp, Artificial Sequence
  Synthetic
ctgtgtagtttgtgctgta

SEQ ID NO: 8 - DNA, 19 bp, Artificial Sequence
  Synthetic
agcattacgccaatagaaa

SEQ ID NO: 9 - DNA, 19 bp, Artificial Sequence
  Synthetic
gctacaaactcctaaggaa

SEQ ID NO: 10 - DNA, 18 bp, Artificial Sequence
  Synthetic
ctggagattcctaccttt

SEQ ID NO: 11 - DNA, 19 bp, Artificial Sequence
  Synthetic
agcagcaatggcaatgata

SEQ ID NO: 12 - DNA, 19 bp, Artificial Sequence
  Synthetic
aagtttgtgatccactgta

SEQ ID NO: 13 - DNA, 19 bp, Artificial Sequence
  Synthetic
ggaccagatcagtaatata

SEQ ID NO: 14 - DNA, 19 bp, Artificial Sequence
  Synthetic
attatttaatgggctattt

SEQ ID NO: 15 - DNA, 19 bp, Artificial Sequence
  Synthetic
gctagtatgttggaagtta

SEQ ID NO: 16 - DNA, 19 bp, Artificial Sequence
  Synthetic
ctttgaatgggcgagtgat

SEQ ID NO: 17 - DNA, 19 bp, Artificial Sequence
  Synthetic
gcaagaagaaggtctggtt

SEQ ID NO: 18 - DNA, 19 bp, Artificial Sequence
  Synthetic
cccatagggagctgatcgt

SEQ ID NO: 19 - DNA, 19 bp, Artificial Sequence
  Synthetic
ctaccagttggaacactta

SEQ ID NO: 20 - DNA, 19 bp, Artificial Sequence
  Synthetic
ctcgccatctgcatccatc

SEQ ID NO: 21 - DNA, 19 bp, Artificial Sequence
  Synthetic
aggagatcatccagtacaa

SEQ ID NO: 22 - DNA, 19 bp, Artificial Sequence
  Synthetic
ctggcccaggctgaagttt

SEQ ID NO: 23 - DNA, 19 bp, Artificial Sequence
  Synthetic
gggaggaagctgatgaata

SEQ ID NO: 24 - DNA, 19 bp, Artificial Sequence
  Synthetic
cacatgaccttaagattat

SEQ ID NO: 25 - DNA, 21 bp, Artificial Sequence
  Synthetic
agacacgtttgagtccatgcc

SEQ ID NO: 26 - DNA, 21 bp, Artificial Sequence
  Synthetic
atcccacgtccgtagaaaggt

SEQ ID NO: 27 - DNA, 22 bp, Artificial Sequence
  Synthetic
ttcttcctcatcttcctcgtcg

SEQ ID NO: 28 - DNA, 22 bp, Artificial Sequence
  Synthetic
cgtccccacacactttacacag

SEQ ID NO: 29 - DNA, 21 bp, Artificial Sequence
  Synthetic
ggcaacaacccaatgagtggt

SEQ ID NO: 30 - DNA, 22 bp, Artificial Sequence
  Synthetic
atgccgatctttgatgctcatg

SEQ ID NO: 31 - DNA, 22 bp, Artificial Sequence
  Synthetic
ccgtggacctgatcatcaagag

SEQ ID NO: 32 - DNA, 21 bp, Artificial Sequence
  Synthetic
tcgatgacgtggacggatact

SEQ ID NO: 33 - DNA, 20 bp, Artificial Sequence
  Synthetic
aagatctcgtggccaatgga

SEQ ID NO: 34 - DNA, 20 bp, Artificial Sequence
  Synthetic
aggtttgcggatcaaaccaa

SEQ ID NO: 35 - DNA, 20 bp, Artificial Sequence
  Synthetic
ctttgtgcccacggttatca

SEQ ID NO: 36 - DNA, 21 bp, Artificial Sequence
  Synthetic
agtggtccctgtttgtctcca

SEQ ID NO: 37 - DNA, 21 bp, Artificial Sequence
  Synthetic
tgcaaggaaaggaagttggtg

SEQ ID NO: 38 - DNA, 21 bp, Artificial Sequence
  Synthetic
ttcgagccttgatgacagcag

SEQ ID NO: 39 - DNA, 20 bp, Artificial Sequence
  Synthetic
tggtcattgaccgcaaggtt

SEQ ID NO: 40 - DNA, 21 bp, Artificial Sequence
  Synthetic
aggcatccgttgcatcttctc

SEQ ID NO: 41 - DNA, 21 bp, Artificial Sequence
  Synthetic
gccattgtggtgactgatgga

SEQ ID NO: 42 - DNA, 21 bp, Artificial Sequence
  Synthetic
tcatccctccgcaagctgtat

SEQ ID NO: 43 - DNA, 22 bp, Artificial Sequence
  Synthetic
gcatggccaaacctaacatgat

SEQ ID NO: 44 - DNA, 21 bp, Artificial Sequence
  Synthetic
cctggcccagtatgaaggaaa

SEQ ID NO: 45 - DNA, 22 bp, Artificial Sequence
  Synthetic
ctgtccaccttccagcagatgt

(19) United States
(12) Patent Application Publication
(10) Pub. No.: US 2010/0267803 A1
(43) Pub. Date: Oct. 21, 2010

(54) Regulators Of Fat Metabolism As Anti-Cancer Targets

(75) Inventors: Douglas S. Conklin, Niskayuna, NY (US); Antonis Kourtidis, Saint Augustine, FL (US)

Correspondence Address: MEDLEN & CARROLL, LLP;Suite 350, 101 Howard Street, San Francisco, CA, 94105, US

(73) Assignee: The Research Foundation of State University Of New York

(21) Appl. No.: 12/613,953

(22) Filed: Nov. 6, 2009

Related U.S. Application Data
(60) Provisional application No. 61/112,497, filed on Nov. 7, 2008.

(51) Int. Cl. A61K 31/7105; C12Q 1/25; C12Q 1/48; C12Q 1/02; A61P 35/00

(52) U.S. Cl. 514/44 A; 435/4; 435/15; 435/29

(57) ABSTRACT

Embodiments of the invention provide methods of identifying agents that reduce or prevent the proliferation of breast cancer cells, or kill them, in particular by interfering with the expression of the transcription factors NR1D1 and PPARγ or the expression of genes whose transcription they activate or the activity of the proteins translated from those transcripts.

### STATEMENT REGARDING FEDERALLY SPONSORED RESEARCH OR DEVELOPMENT

The research described in this application was supported by the U.S. Army Medical Research Acquisition Activitry, contract number W8IWXH-04-1-0474. The United States government may have certain rights in the invention.

### FIELD

Embodiments of the invention find application in the field of cancer therapy.

### BACKGROUND

The overexpression of proteins and altered cellular physiology that result from gene amplification are a significant cause of the tumorigenic nature of cancer cells. Overexpression of the ERBB2 (Her-2/neu) oncogene occurs in nearly 30% of breast cancers and is a prognostic indicator for aggressive disease and reduced survival¹. Although therapies have been developed that effectively target ERBB2, there is still high recurrence of the disease after these treatments² suggesting that factors in addition to ERBB2 itself must contribute to the aggressiveness and poor therapy response in these tumours. Transcriptional profiling meta-analyses have found that ERBB2 is one of approximately 150 genes that are overexpressed in this tumour type³⁻⁵. There is a need to identify which of them confer survival capability in order to provide targets against which anti-cancer agents may be discovered, designed and developed.

### SUMMARY

In one embodiment, the invention provides a method of treating cancer, the method comprising:
a) providing a subject with breast cancer cells and an inhibitor of a gene encoding a protein associated with adipogenesis;
b) treating said subject with said inhibitor.

In a preferred embodiment, the protein of the method is a transcriptional regulator of fat synthesis and storage. In one embodiment, the protein is NR1D1 (RevErb-alpha). In another embodiment, the protein is PBP.

In one embodiment, the inhibitor of the method comprises a short hairpin RNA of SEQ ID NO.1. In another embodiment, the short hairpin RNA is of SEQ ID NO. 2.

In one embodiment, the method of treating with the RNA results in reduced proliferation of said breast cancer cells.

In another embodiment, the invention provides a method of treating cancer, comprising:
a) providing a subject with breast cancer cells and an inhibitor of a gene encoding a protein associated with lipid metabolism, and
b) treating said subject with said inhibitor.

In a preferred embodiment of the method wherein a protein associated with lipid metabolism is inhibited, the protein is selected from The method of Claim 7, wherein said protein associated with adipogenesis is selected from a fatty acid synthase and a fatty acid desaturase. In one embodiment of the method, the inhibitor comprises a short hairpin RNA of SEQ. ID NO:3. In another embodiment, the inhibitor comprises a short hairpin RNA of SEQ. ID NO:4. In preferred embodiments, the treating with the RNA of SEQ. ID NO.3 or SEQ ID NO.4 results in reduced proliferation of said breast cancer cells.

In one embodiment, the invention provides a method of identifying a test agent that affects the expression of a transcription factor selected from the group consisting of NR1D1 or PPARγ, the method comprising: (i) providing a first and a second cell expressing the transcription factor, (ii) contacting the first cell with the test agent, (iii) contacting the second cell with a control agent, (iii) measuring expression of the transcription factor in the first and second cells, and (iv) comparing the amount of expression of the transcription factor in the first and second cells to determine whether or not the test agent promotes or inhibits the expression of the transcription factor.

In one embodiment, the invention provides a method of identifying a test agent that affects the expression of a protein translated from the message transcribed under the influence of the transcription factor, the method comprising: (i) providing a first and a second cell expressing the transcription factor, (ii) contacting the first cell with the test agent, (iii) contacting the second cell with control agent, (iii) measuring expression of the protein in the first and second cells, and (iv) comparing the amount of expression of the protein in the first and second cells to determine whether or not the test agent promotes or inhibits the expression of the protein.

In one embodiment, the invention provides a method of identifying a test agent that affects the activity of a protein translated from the message transcribed under the influence of the transcription factor, the method comprising: (i) providing a first and a second amount of the protein, (ii) contacting the first and second amounts with a substrate of the protein under conditions in which a product forms, expressing the transcription factor, (ii) contacting the first protein amount with the test agent, (iii) contacting the second protein amount with a control agent, (iii) measuring a first rate of formation of the product by the first protein amount and a second rate of production the second protein amount, and (iv) comparing the first and second rates to determine whether or not the test agent promotes or inhibits the activity of the protein. In some embodiments, the amounts are in vitro. In some embodiments, the amounts are in cell-free systems. In some embodiments, the amounts are in cells. In one embodiment, the invention.

### BRIEF DESCRIPTION OF THE DRAWINGS

FIG. 1 An RNAi screen targeting genes overexpressed in breast cancer. (a) Three transfection mixes were produced for each shRNA and each was transfected into triplicate wells of BT474 cells. AlamarBlue was used to monitor cell proliferation and viability. The averages of nine parallel cultures were calculated for each shRNA, normalized to transfection efficiency, expressed as % of the control shRNA (luciferase) and sorted on the basis of effect (top panel). shRNAs that produced more than (++++) or nearly (+++) a 50% decrease in proliferation and a selection of other significant hits that had a moderate effect (++) are presented in a list (bottom panel). (b) The genetic and functional linkage of NR1D1 and PBP. Mb refers to map position on chromosome 17. (c) ERBB2-overexpressing (OEX) and ERBB2 non-overexpressing (non-OEX) breast cancer cell lines as well as the (non-OEX) HMEC and HEK 293 cells were transfected and assayed as in (a). Effects of each shRNA on each cell line were subjected to hierarchical cluster analysis and displayed using Treeview. Genes clustered with ERBB2 are boxed.

FIG. 2 NR1D1 and PBP are necessary for survival of ERBB2-positive cells. (a) PBP and NR1D1 mRNA levels of BT474 cells transfected with luciferase (control), PBP or NR1D1 shRNAs for 48 h, analyzed by qRT-PCRs. (Error bars are s.d from 3 experiments; ***P<0.003, **P=0.01, *P=0.02). (b) BMAL1 luciferase reporter activity of BT474 cells transfected with either empty vector (control), or NR1D1 shRNA for 48 h.

Results are shown as percentage of control. (**P=0.03). (c) BT474 cells were transfected with luciferase (control), PBP and NR1D1 shRNAs and co-transfected with GFP. Green cells were counted at intervals and expressed as percentage of control. (Error bars are s.d from 3 individual experiments; *P<0.08, **P<0.03, ***P<0.005). (d) BT474 cells were transfected with luciferase (control), PBP or NR shRNAs for 48 h. Immunofluorescence was performed for cleaved Caspase-3 signal; cell nuclei were stained with Hoechst 33243. (e) Cells from (e) were counted for cleaved Caspase-3 signal. (Error bars are s.d from 3 individual experiments; ***P<0.0003). (f) BT474 cells were transfected with luciferase (control) PBP, NR1D1 or PBP and NR1D1 shRNAs together and co-transfected with GFP. Transfected cells were counted at 24 h and 72 h and the 72 h-to-24 h ratio for each shRNA was calculated and expressed as % of the control shRNA (luciferase). (Error bars are s.d from 3 individual experiments; **P=0.01, ***P<0.002). (g) Cells from (f) were counted for cleaved Caspase-3 signal. (Error bars are s.d from 3 individual experiments; ***P<0.0001). (h) ERBB2 mRNA levels of BT474 cells transfected with luciferase (control), PBP or NR1D1 shRNAs for 48 h, analyzed by qRT-PCRs. (Error bars are s.d from 3 individual experiments; ***P=0.0001).

FIG. 3 PPAR. inhibition impacts NR1D1 expression and ERBB2-positive cells viability. (a) Cell counts of BT474, MDA-MB-361, MCF-7 and HMEC cells treated with vehicle, 10 and 20 μM of the PPAR. antagonist GW9662 for 72 hrs. Results represented as % of control (vehicle). (Error bars are s.d from 3 individual experiments; *P>0.2, **P<0.05, ***P<0.002). (b) BT474 cells treated with vehicle or 20 μM of GW9662 for 48 h were immuno-stained for cleaved Caspase-3. (c) Quantification of Caspase-3 signal. (Error bars are s.d from 3 individual experiments; ***P=0.002). (d) aP2 and NR1D1 mRNA levels of BT474 cells treated with vehicle (control) and 20 μM of GW9662 for 48 h, analyzed by qRT-PCRs. (Error bars are s.d from 3 experiments; ***P<0.0006. (e) BMAL1-luc activity of BT474 cells treated with vehicle or 20 μM of GW9662 for 48 h. (Error bars are s.d from 3 individual experiments; **P=0.03). (f) Total ERBB2 western blot of BT474 cells treated with vehicle or 20 μM GW9662 for 72h. GAPDH was used as control.

FIG. 4 NR1D1 and PBP coordinate fatty acid metabolism in breast cancer cells. (a) BT474, MDA-MB-361, MCF-7 and HMEC cells were stained with Oil-Red O lipid and hematoxylin nuclear stains. (b) Fat stores were quantified by BODIPY 493/503 lipid probe staining (Error bars are s.d from 3 individual experiments). (c) FASN, ACLY, ACACA and FADS2 mRNA levels of BT474 cells treated with luciferase (control), PBP or NR1D1 shRNAs, or with vehicle and 20 μM of GW9662 for 48 h, analyzed by qRT-PCRs. (Error bars are s.d from 3 experiments; ***P<0.001, **P=0.005) (d) BT474 cells were transfected with FASN, ACLY and ACACA shRNAs and co-transfected with GFP. Transfected cells were counted at 24 h and 72 h and the 72h-to-24 h ratio for each shRNA was calculated and expressed as % of the control shRNA (luciferase). (Error bars are s.d from 3 individual experiments; **P<0.01, ***P<0.002). (e) BODIPY images and (f) quantification of BT474 cells transfected with luciferase (control), PBP or NR1D1 shRNAs and co-transfected with DsRed (Error bars are s.d from 3 individual experiments; ***P<0.03). Arrows indicate transfected cells.

FIG. 5. (a) MDH1 and ME1 mRNA levels of BT474 cells treated with luciferase (control), PBP or NR1D1 shRNAs for 48 h, analyzed by qRT-PCRs. (Error bars are s.d from 3 experiments; **P<0.002 (b) BT474 and MCF-7 cells were transfected with luciferase (control) MDH1 and ME1 shRNAs (Error bars are s.d from 3 individual experiments; ***P=0.0003). (c) BT474 cells) assayed for cleaved Caspase-3 signal by immunofluorescence 48 h after transfection; cell nuclei were stained with Hoechst 33243. (d) Quantification of cleaved Caspase-3 signal from cells in (c). (Error bars are s.d from 3 individual experiments; ***P<0.005). (e) Enzymes involved in fatty acid synthesis and storage that are regulated by NR1D1 and PBP and their proposed contribution to energy production necessary for survival of ERBB2-positive breast cancer cells.

FIG. S1 shows that NR1D1 and PbP shRNAs result in increased cell death and apoptosis of BT474 cells.

FIG. S2 shows that several NR1D1 and PbP shRNAs result in increased cell death and apoptosis specifically of BT474 cells.

FIG. S3 shows that PPARγ inhibition produces effects similar to NR1D1 and PBP downregulation.

FIG. S4 shows that ERBB2-positive cells have high levels of fats.

FIG. S5 shows that supplementation of insulin does not affect fat metabolism in BT474 cells.

FIG. S6 shows that alternative carbon sources lead to decreased fat stores but not cell death.

Table S1 lists several shRNA sequences used.

Table S2 shows the primer pairs used for qRT-PCR

### DEFINITIONS

To facilitate the understanding of this invention a number of terms (set off in quotation marks in this Definitions section) are defined below. Terms defined herein (unless otherwise specified) have meanings as commonly understood by a person of ordinary skill in the areas relevant to the present invention. As used in this specification and its appended claims, terms such as “a”, “an” and “the” are not intended to refer to only a singular entity, but include the general class of which a specific example may be used for illustration, unless the context dictates otherwise. The terminology herein is used to describe specific embodiments of the invention, but their usage does not delimit the invention, except as outlined in the claims.

The phrase “chosen from A, B, and C” as used herein, means selecting one or more of A, B, C.

As used herein, absent an express indication to the contrary, the term “or” when used in the expression “A or B,” where A and B refer to a composition, disease, product, etc., means one or the other, or both. As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the context clearly dictates otherwise. For example, a method comprising steps a, b, and c encompasses, for example, a method of performing steps in the order of steps a, c, and b, the order of steps c, b, and a, and the order of steps c, a, and b, etc.

Unless otherwise indicated, all numbers expressing quantities of ingredients, properties such as molecular weights, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

The term “not” when preceding, and made in reference to, any particularly named molecule (mRNA, etc.) or phenomenon (such as biological activity, biochemical activity, etc.) means that only the particularly named molecule or phenomenon is excluded.

The term “altering” and grammatical equivalents as used herein in reference to the level of any substance and/or phenomenon refers to an increase and/or decrease in the quantity of the substance and/or phenomenon, regardless of whether the quantity is determined objectively, and/or subjectively.

The terms “increase,” “elevate,” “raise,” and grammatical equivalents when used in reference to the level of a substance and/or phenomenon in a first sample relative to a second sample, mean that the quantity of the substance and/or phenomenon in the first sample is higher than in the second sample by any amount that is statistically significant using any art-accepted statistical method of analysis. In one embodiment, the increase may be determined subjectively, for example when a patient refers to their subjective perception of disease symptoms, such as pain, clarity of vision, etc. In another embodiment, the quantity of the substance and/or phenomenon in the first sample is at least 10% greater than the quantity of the same substance and/or phenomenon in a second sample. In another embodiment, the quantity of the substance and/or phenomenon in the first sample is at least 25% greater than the quantity of the same substance and/or phenomenon in a second sample. In yet another embodiment, the quantity of the substance and/or phenomenon in the first sample is at least 50% greater than the quantity of the same substance and/or phenomenon in a second sample. In a further embodiment, the quantity of the substance and/or phenomenon in the first sample is at least 75% greater than the quantity of the same substance and/or phenomenon in a second sample. In yet another embodiment, the quantity of the substance and/or phenomenon in the first sample is at least 90% greater than the quantity of the same substance and/or phenomenon in a second sample. Alternatively, a difference may be expressed as an “n-fold” difference.

The terms “reduce,” “inhibit,” “diminish,” “suppress,” “decrease,” and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, clarity of vision, etc. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample. Alternatively, a difference may be expressed as an “n-fold” difference.

A number of terms herein relate to cancer. “Cancer” is intended herein to encompass all forms of abnormal or improperly regulated reproduction of cells in a subject. “Subject” and “patient” are used herein interchangeably, and a subject may be any mammal but is preferably a human. A “reference subject” herein refers to an individual who does not have cancer. The “reference subject” thereby provides a basis to which another cell (for example a cancer cell) can be compared.).

The growth of cancer cells (“growth” herein referring generally to cell division but also to the growth in size of masses of cells) is characteristically uncontrolled or inadequately controlled, as is the death (“apoptosis”) of such cells. Local accumulations of such cells result in a tumor. More broadly, and still denoting “tumors” herein are accumulations ranging from a cluster of lymphocytes at a site of infection to vascularized overgrowths, both benign and malignant. A “malignant” tumor (as opposed to a “benign” tumor) herein comprises cells that tend to migrate to nearby tissues, including cells that may travel through the circulatory system to invade or colonize tissues or organs at considerable remove from their site of origin in the “primary tumor,” so-called herein. Metastatic cells are adapted to penetrate blood vessel wells to enter (“intravasate”) and exit (“extravasate”) blood vessels. Tumors capable of releasing such cells are also referred to herein as “metastatic.” The term is used herein also to denote any cell in such a tumor that is capable of such travel, or that is en route, or that has established a foothold in a target tissue. For example, a metastatic breast cancer cell that has taken root in the lung is referred to herein as a “lung metastasis.” Metastatic cells may be identified herein by their respective sites of origin and destination, such as “breast-to-bone metastatic.” In the target tissue, a colony of metastatic cells can grow into a “secondary tumor,” so called herein.

Primary tumors are thought to derive from a benign or normal cell through a process referred to herein as “cancer progression.” According to this view, the transformation of a normal cell to a cancer cell requires changes (usually many of them) in the cell's biochemistry. The changes are reflected clinically as the disease progresses through stages. Even if a tumor is “clonogenic” (as used herein, an accumulation of the direct descendants of a parent cell), the biochemistry of the accumulating cells changes in successive generations, both because the expression of the genes (controlled by so-called “epigenetic” systems) of these cells becomes unstable and because the genomes themselves change. In normal somatic cells, the genome (that is, all the genes of an individual) is stored in the chromosomes of each cell (setting aside the mitochondrial genome). The number of copies of any particular gene is largely invariant from cell to cell. By contrast, “genomic instability” is characteristic of cancer progression. A genome in a cancer cell can gain (“genomic gain”) or lose (“genomic loss”) genes, typically because an extra copy of an entire chromosome appears (“trisomy”) or a region of a chromosome replicates itself (“genomic gain” or, in some cases, “genomic amplification”) or drops out when the cell divides. Thus, the “copy number” of a gene or a set of genes, largely invariant among normal cells, is likely to change in cancer cells (referred to herein as a “genomic event”), which affects the total expression of the gene or gene set and the biological behavior (“phenotype”) of descendent cells. Thus, in cancer cells, “gene activity” herein is determined not only by the multiple “layers” of epigenetic control systems and signals that call forth expression of the gene but by the number of times that gene appears in the genome. The term “epigenetic” herein refers to any process in an individual that, in operation, affects the expression of a gene or a set of genes in that individual, and stands in contrast to the “genetic” processes that govern the inheritance of genes in successive generations of cells or individuals.

Certain regions of chromosomes, depending upon the specific type of cancer, have proven to be hot spots for genomic gain inasmuch as increases in copy number in the genomes of cells from multiple donors tend to occur in one or a few specific regions of a specific chromosome. Such hot spots are referred to herein as sites of “recurrent genomic gain.” The term is to be distinguished from “recurrent cancer,” which refers to types of cancer that are likely to recur after an initial course of therapy, resulting in a “relapse.”

A number of terms herein relate to methods that enable the practitioner to examine many distinct genes at once. By these methods, sets of genes (“gene sets”) have been identified wherein each set has biologically relevant and distinctive properties as a set. Devices (which may be referred to herein as “platforms”) in which each gene in a significant part of an entire genome is isolated and arranged in an array of spots, each spot having its own “address,” enable one to detect, quantitatively, many thousands of the genes in a cell. More precisely, these “microarrays” typically detect expressed genes (an “expressed” gene is one that is actively transmitting its unique biochemical signal to the cell in which the gene resides). Microarray data, inasmuch as they display the expression of many genes at once, permit the practitioner to view “gene expression profiles” in a cell and to compare those profiles cell-to-cell to perform so-called “comparative analyses of expression profiles.” Such microarray-based “expression data” are capable of identifying genes that are “overexpressed” (or underexpressed) in, for example, a disease condition. An overexpressed gene may be referred to herein as having a high “expression score.”

The aforementioned methods for examining gene sets employ a number of well-known methods in molecular biology, to which references are made herein. A gene is a heritable chemical code resident in, for example, a cell, virus, or bacteriophage that an organism reads (decodes, decrypts, transcribes) as a template for ordering the structures of biomolecules that an organism synthesizes to impart regulated function to the organism. Chemically, a gene is a heteropolymer comprised of subunits (“nucleotides”) arranged in a specific sequence. In cells, such heteropolymers are deoxynucleic acids (“DNA”) or ribonucleic acids (“RNA”). DNA forms long strands. Characteristically, these strands occur in pairs. The first member of a pair is not identical in nucleotide sequence to the second strand, but complementary. The tendency of a first strand to bind in this way to a complementary second strand (the two strands are said to “anneal” or “hybridize”), together with the tendency of individual nucleotides to line up against a single strand in a complementarily ordered manner accounts for the replication of DNA.

Experimentally, nucleotide sequences selected for their complementarity can be made to anneal to a strand of DNA containing one or more genes. A single such sequence can be employed to identify the presence of a particular gene by attaching itself to the gene. This so-called “probe” sequence is adapted to carry with it a “marker” that the investigator can readily detect as evidence that the probe struck a target. As used herein, the term “marker” relates to any surrogate the artisan may use to “observe” an event or condition that is difficult or impossible to detect directly. In some contexts herein, the marker is said to “target” the condition or event. In other contexts, the condition or event is referred to as the target for the marker. Sequences used as probes may be quite small (e.g., “oligonucleotides” of <20 nucleotides) or quite large (e.g., a sequence of 100,000 nucleotides in DNA from a “bacterial artificial chromosome” or “BAC”). A BAC is a bacterial chromosome (or a portion thereof) with a “foreign” (typically, human) DNA fragment inserted in it. BACs are employed in a technique referred to herein as “fluorescence in situ hybridization” or “FISH.” A BAC or a portion of a BAC is constructed that has (1) a sequence complementary to a region of interest on a chromosome and (2) a marker whose presence is discernible by fluorescence. The chromosomes of a cell or a tissue are isolated (on a glass slide, for example) and treated with the BAC construct. Excess construct is washed away and the chromosomes examined microscopically to find chromosomes or, more particularly, identifiable regions of chromosomes that fluoresce.

Alternatively, such sequences can be delivered in pairs selected to hybridize with two specific sequences that bracket a gene sequence. A complementary strand of DNA then forms between the “primer pair.” In one well-known method, the “polymerase chain reaction” or “PCR,” the formation of complementary strands can be made to occur repeatedly in an exponential amplification. A specific nucleotide sequence so amplified is referred to herein as the “amplicon” of that sequence. “Quantitative PCR” or “qPCR” herein refers to a version of the method that allows the artisan not only to detect the presence of a specific nucleic acid sequence but also to quantify how many copies of the sequence are present in a sample, at least relative to a control. As used herein, “qRTPCR” may refer to “quantitative real-time PCR,” used interchangeably with “qPCR” as a technique for quantifying the amount of a specific DNA sequence in a sample. However, if the context so admits, the same abbreviation may refer to “quantitative reverse transcriptase PCR,” a method for determining the amount of messenger RNA present in a sample. Since the presence of a particular messenger RNA in a cell indicates that a specific gene is currently active (being expressed) in the cell, this quantitative technique finds use, for example, in gauging the level of expression of a gene.

Collectively, the genes of an organism constitute its genome. The term “genomic DNA” may refer herein to the entirety of an organism's DNA or to the entirety of the nucleotides comprising a single gene in an organism. A gene typically contains sequences of nucleotides devoted to coding (“exons”), and non-coding sequences that contribute in one way or another to the decoding process (“introns”).

The term “gene” refers to a nucleic acid (e.g., DNA) comprising covalently linked nucleotide monomers arranged in a particular sequence that comprises a coding sequence necessary for the production of a polypeptide or precursor or RNA (e.g., tRNA, siRNA, rRNA, etc.). The polypeptide can be encoded by a full-length coding sequence or by any portion of the coding sequence so long as the desired activities or functional properties (e.g., enzymatic activity, ligand binding, signal transduction, etc.) of the full-length or fragment are retained. The term also encompasses the coding region together with the sequences located adjacent to the coding region on both the 5′ and 3′ ends, such that the gene corresponds to the length of the full-length mRNA (also referred to as “pre-mRNA,” “nuclear RNA,” or “primary transcript RNA”) transcribed from it. The sequences that are located 5′ of the coding region and are present on the mRNA are referred to as 5′ untranslated sequences. The sequences that are located 3′ or downstream of the coding region and that are present on the mRNA are referred to as 3′ untranslated sequences. The term “gene” encompasses both cDNA (the coding region(s) only) and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

Encoding in DNA (and messenger RNA) is accomplished by 3-membered nucleotide sequences called “codons.” Each codon encrypts an amino acid, and the sequence of codons encrypts the sequence of amino acids that identifies a particular protein. The code for a given gene is embedded in a (usually) much longer nucleotide sequence and is distinguishable to the cell's decoding system from the longer sequence by a “start codon” and a “stop” codon. The decoding system reads the sequence framed by these two codons (the so-called “open reading frame”). The readable code is transcribed into messenger RNA which itself comprises sites that ensure coherent translation of the code from nucleic acid to protein. In particular, the open reading frame is delimited by a so-called “translation initiation” codon and “translation termination” codon.

The term “plasmid” as used herein, refers to a small, independently replicating, piece of DNA. Similarly, the term “naked plasmid” refers to plasmid DNA devoid of extraneous material typically used to effect transfection. As used herein, a “naked plasmid” refers to a plasmid substantially free of calcium-phosphate, DEAE-dextran, liposomes, and/or polyamines. As used herein, the term “purified” refers to molecules (polynucleotides or polypeptides) that are removed from their natural environment, isolated or separated. “Purified” molecules are at least 50% free, preferably at least 75% free, and more preferably at least 90% free from other components with which they are naturally associated.

The term “recombinant DNA” refers to a DNA molecule that is comprised of segments of DNA joined together by means of molecular biology techniques. Similarly, the term “recombinant protein” refers to a protein molecule that is expressed from recombinant DNA.

The term “fusion protein” as used herein refers to a protein formed by expression of a hybrid gene made by combining two gene sequences. Typically this is accomplished by cloning a cDNA into an expression vector in frame (i.e., in an arrangement that the cell can transcribe as a single mRNA molecule) with an existing gene. The fusion partner may act as a reporter (e.g., βgal) or may provide a tool for isolation purposes (e.g., GST).

Where an amino acid sequence is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Rather the terms “amino acid sequence” and “protein” encompass partial sequences, and modified sequences.

The term “wild type” refers to a gene or gene product that has the characteristics of that gene or gene product when isolated from a naturally occurring source. A wild type gene is the variant most frequently observed in a population and is thus arbitrarily designed the “normal” or “wild-type” form of the gene

In contrast, the terms “modified,” “mutant,” and “variant” (when the context so admits) refer to a gene or gene product that displays modifications in sequence and or functional properties (i.e., altered characteristics) when compared to the wild-type gene or gene product. In some embodiments, the modification comprises at least one nucleotide insertion, deletion, or substitution.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to reduction in binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target. When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “competes for binding” when used in reference to a first and a second polypeptide means that the first polypeptide with an activity binds to the same substrate as does the second polypeptide with an activity. In one embodiment, the second polypeptide is a variant of the first polypeptide (e.g., encoded by a different allele) or a related (e.g., encoded by a homolog) or dissimilar (e.g., encoded by a second gene having no apparent relationship to the first gene) polypeptide. The efficiency (e.g., kinetics or thermodynamics) of binding by the first polypeptide may be the same as or greater than or less than the efficiency of substrate binding by the second polypeptide. For example, the equilibrium binding constant (K_{D}) for binding to the substrate may be different for the two polypeptides.

As used herein, the term “hybridization” refers to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with 85-100% identity, preferably 70-100% identity). With medium stringency conditions, nucleic acid base pairing will occur between nucleic acids with an intermediate frequency of complementary base sequences (e.g., hybridization under “medium stringency” conditions may occur between homologs with 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution comprising 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 100 to about 1000 nucleotides in length is employed.

“Medium stringency conditions” when used in reference to nucleic acid hybridization comprise conditions equivalent to binding or hybridization at 42° C. in a solution comprising 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 100 to about 1000 nucleotides in length is employed.

“Low stringency conditions” comprise conditions equivalent to binding or hybridization at 42° C. in a solution comprising 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42° C. when a probe of about 100 to about 1000 nucleotides in length is employed.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 85% to 95% homology to the first nucleic acid sequence, then another hybridization condition is said to be equivalent to the hybridization condition of interest if this other hybridization condition also results in hybridization of the first nucleic acid sequence with the other nucleic acid sequences that have from 85% to 95% homology to the first nucleic acid sequence.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math., 2: 482, 1981) by the homology alignment algorithm of Needleman and Wunsch (Needleman and Wunsch, J. Mol. Biol., 48:443, 1970), by the search for similarity method of Pearson and Lipman (Pearson and Lipman, Proc. Natl. Acad. Sci., U.S.A., 85:2444, 1988), by computerized implementations of these algorithms (GAP, BESTFIT, FASTA, and TFASTA in the Wisconsin Genetics Software Package Release 7.0, Genetics Computer Group, Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having acidic side chains is glutamic acid and aspartic acid; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

“Amplification” is used herein in two different ways. A given gene typically appears in a genome once, on one chromosome. Since chromosomes in somatic cells of eukaryotes are in general paired, two copies or alleles of each gene are found. In some conditions, such as cancer, replication of chromosome pairs during cell division is disturbed so that multiple copies of a gene or chromosome accrue over successive generations. The pheonomenon is referred to generally (and herein) as “amplification.”

In the context of molecular biological experimentation, the term is used differently. Experimentally, “amplification” is used in relation to a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under the conditions in which they are used, will process only specific sequences of nucleic acids in a heterogeneous mixture of nucleic acids. In particular, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences.

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular sequences. It is contemplated that any probe used in the present invention will be labelled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the method of Mullis (U.S. Pat. Nos. 4,683,195, 4,683,202, and 4,965,188, hereby incorporated by reference), that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

As used herein, the terms “restriction endonucleases” and “restriction enzymes” refer to bacterial enzymes, each of which cut double-stranded DNA at or near a specific nucleotide sequence.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding gene includes, by way of example, such nucleic acid in cells ordinarily expressing gene where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

The terms “fragment” and “portion” when used in reference to a nucleotide sequence (as in “a portion of a given nucleotide sequence”) refers to partial segments of that sequence. The fragments may range in size from four nucleotides to the entire nucleotide sequence minus one nucleotide (10 nucleotides, 20, 30, 40, 50, 100, 200, etc.).

Similarly, the terms “fragment” and “portion” when used in reference to a polypeptide sequence refers to partial segments of that sequence. In some embodiments, the portion has an amino-terminal and/or carboxy-terminal deletion as compared to the native protein, but where the remaining amino acid sequence is identical to the corresponding positions in the amino acid sequence deduced from a full-length cDNA sequence. Fragments are preferably at least 4 amino acids long, more preferably at least 50 amino acids long, and most preferably at least 50 amino acids long or longer (the entire amino acid sequence minus on amino acid). In particularly preferred embodiments, the portion comprises the amino acid residues required for intermolecular binding of the compositions of the present invention with its various ligands and/or substrates.

As used herein the term “portion” when in reference to a protein (as in “a portion of a given protein”) refers to fragments of that protein. The fragments may range in size from four consecutive amino acid residues to the entire amino acid sequence minus one amino acid

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets which specify stop codons (i.e., TAA, TAG, TGA

The term “recombinant DNA molecule” as used herein refers to a DNA molecule that is comprised of segments of DNA joined together by means of molecular biological techniques. Similarly, the term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences, that is the native protein contains only those amino acids found in the protein as it occurs in nature. A native protein may be produced by recombinant means or may be isolated from a naturally occurring source.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58, 1989).

The term “Northern blot,” as used herein refers to the analysis of RNA by electrophoresis of

RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp 7.39-7.52, 1989).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies

As used herein, the term “transgenic” refers to a cell or organism whose genome has been heritably altered by genetically engineering into the genome a gene (“transgene”) not normally part of it or removing from it a gene ordinarily present (a “knockout” gene). The “transgene” or “foreign gene” may be placed into an organism by introducing it into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term host cell refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell in the sense that the foreign DNA will be passed on to daughter cells. The term encompasses transfections of foreign DNA into the cytoplasm only. In general, however, the foreign DNA reaches the nucleus of the transfected cell and persists there for several days. During this time the foreign DNA is subject to the regulatory controls that govern the expression of endogenous genes in the chromosomes. The term “transient transfectant” refers to cells that have taken up foreign DNA but have failed to integrate this DNA. The term “transient transfection” encompasses transfection of foreign DNA into the cytoplasm only

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of is modified to optimize conditions for particular types of cells. The art is well aware of these numerous modifications.

A “composition comprising a given polynucleotide sequence” as used herein refers broadly to any composition containing the given polynucleotide sequence. Such compositions may be employed as hybridization probes, typically in an aqueous solution containing salts (e.g., NaCl), detergents (e.g., SDS), and other components (e.g., Denhardt's solution, dry milk, salmon sperm DNA, etc.).

The terms “N-terminus” “NH₂ -terminus” and “amino-terminus” refer to the amino acid residue corresponding to the methionine encoded by the start codon (e.g., position or residue 1). In contrast the terms “C-terminus” “COOH-terminus” and “carboxy terminus” refer to the amino acid residue encoded by the final codon (e.g., last or final residue prior to the stop codon).

The term “conservative substitution” as used herein refers to a change that takes place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine). Whether a change in the amino acid sequence of a peptide results in a functional homolog can be readily determined by assessing the ability of the variant peptide to function in a fashion similar to the wild-type protein. Peptides having more than one replacement can readily be tested in the same manner. In contrast, the term “nonconservative substitution” refers to a change in which an amino acid from one family is replaced with an amino acid from another family (e.g., replacement of a glycine with a tryptophan). Guidance in determining which amino acid residues can be substituted, inserted, or deleted without abolishing biological activity can be found using computer programs (e.g., LASERGENE software, DNASTAR Inc., Madison, Wis.

A peptide sequence and nucleotide sequence may be “endogenous” or “heterologous” (i.e., “foreign”). The term “endogenous” refers to a sequence which is naturally found in the cell or virus into which it is introduced so long as it does not contain some modification relative to the naturally-occurring sequence. The term “heterologous” refers to a sequence which is not endogenous to the cell or virus into which it is introduced. For example, heterologous DNA includes a nucleotide sequence which is ligated to, or is manipulated to become ligated to, a nucleic acid sequence to which it is not ligated in nature, or to which it is ligated at a different location in nature. Heterologous DNA also includes a nucleotide sequence which is naturally found in the cell or virus into which it is introduced and which contains some modification relative to the naturally-occurring sequence. Generally, although not necessarily, heterologous DNA encodes heterologous RNA and heterologous proteins that are not normally produced by the cell or virus into which it is introduced. Examples of heterologous DNA include reporter genes, transcriptional and translational regulatory sequences, DNA sequences which encode selectable marker proteins (e.g., proteins which confer drug resistance), etc. In preferred embodiments, the terms “heterologous antigen” and “heterologous sequence” refer to a non-hepadna virus antigen or amino acid sequence including but not limited to microbial antigens, mammalian antigens and allergen antigens.

The terms “peptide,” “peptide sequence,” “amino acid sequence,” “polypeptide,” and “polypeptide sequence” are used interchangeably herein to refer to at least two amino acids or amino acid analogs which are covalently linked by a peptide bond or an analog of a peptide bond. The term peptide includes oligomers and polymers of amino acids or amino acid analogs. The term peptide also includes molecules which are commonly referred to as peptides, which generally contain from about two (2) to about twenty (20) amino acids. The term peptide also includes molecules which are commonly referred to as polypeptides, which generally contain from about twenty (20) to about fifty amino acids (50). The term peptide also includes molecules which are commonly referred to as proteins, which generally contain from about fifty (50) to about three thousand (3000) amino acids. The amino acids of the peptide may be L-amino acids or D-amino acids. A peptide, polypeptide or protein may be synthetic, recombinant or naturally occurring. A synthetic peptide is a peptide which is produced by artificial means in vitro

The terms “oligosaccharide” and “OS” antigen refer to a carbohydrate comprising up to ten component sugars, either O or N linked to the next sugar. Likewise, the terms “polysaccharide” and “PS” antigen refer to polymers of more than ten monosaccharide residues linked glycosidically in branched or unbranched chains

As used herein, the term “mammalian sequence” refers to synthetic, recombiant or purified sequences (preferably sequence fragments comprising at least one B cell epitope) of a mammal. Exemplary mammalian sequences include cytokine sequence, MHC class I heavy chain sequences, MHC class II alpha and beta chain sequences, and amyloid β-peptide sequences.

The terms “mammals” and “mammalian” refer animals of the class mammalia which nourish their young by fluid secreted from mammary glands of the mother, including human beings. The class “mammalian” includes placental animals, marsupial animals, and monotrematal animals. An exemplary “mammal” may be a rodent, primate (including simian and human) ovine, bovine, ruminant, lagomorph, porcine, caprine, equine, canine, feline, ave, etc. Preferred non-human animals are selected from the order Rodentia.

Preferred embodiments of the present invention are primarily directed to vertebrate (backbone or notochord) members of the animal kingdom.

The terms “patient” and “subject” refer to a mammal that may be treated using the methods of the present invention.

The term “control” refers to subjects or samples which provide a basis for comparison for experimental subjects or samples. For instance, the use of control subjects or samples permits determinations to be made regarding the efficacy of experimental procedures. In some embodiments, the term “control subject” refers to a subject that which receives a mock treatment (e.g., saline alone).

The terms “diluent” and “diluting agent” as used herein refer to agents used to diminish the strength of an admixture. Exemplary diluents include water, physiological saline solution, human serum albumin, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

The terms “carrier” and “vehicle” as used herein refer to usually inactive accessory substances into which a pharmaceutical substance is suspended. Exemplary carriers include liquid carriers (such as water, saline, culture medium, saline, aqueous dextrose, and glycols) and solid carriers (such as carbohydrates exemplified by starch, glucose, lactose, sucrose, and dextrans, anti-oxidants exemplified by ascorbic acid and glutathione, and hydrolyzed proteins.

The term “derived” when in reference to a peptide derived from a source (such as a microbe, cell, etc.) as used herein is intended to refer to a peptide which has been obtained (e.g., isolated, purified, etc.) from the source. Alternatively, or in addition, the peptide may be genetically engineered and/or chemically synthesized.

The terms “operably linked,” “in operable combination,” and “in operable order” as used herein refer to the linkage of nucleic acid sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest.

Similarly, operably linking a nucleic acid sequence encoding a protein of interest means linking the nucleic acid sequence to regulatory and other sequences in a manner such that the protein of interest is expressed. The term also refers to the linkage of amino acid sequences in such a manner so that a functional protein is produced.

The terms “C-terminal portion,” “COOH-terminal portion,” “carboxy terminal portion,” “C-terminal domain,” “COOH-terminal domain,” and “carboxy terminal domain,” when used in reference to an amino acid sequence of interest refer to the amino acid sequence (and portions thereof that is located from approximately the middle of the amino acid sequence of interest to the C-terminal-most amino acid residue of the sequence of interest. The terms “specific binding,” “binding specificity,” and grammatical equivalents thereof when made in reference to the binding of a first molecule (such as a polypeptide, glycoprotein, nucleic acid sequence, etc.) to a second molecule (such as a polypeptide, glycoprotein, nucleic acid sequence, etc.) refer to the preferential interaction between the first molecule with the second molecule as compared to the interaction between the second molecule with a third molecule. Specific binding is a relative term that does not require absolute specificity of binding; in other words, the term “specific binding” does not require that the second molecule interact with the first molecule in the absence of an interaction between the second molecule and the third molecule. Rather, it is sufficient that the level of interaction between the first molecule and the second molecule is higher than the level of interaction between the second molecule with the third molecule. “Specific binding” of a first molecule with a second molecule also means that the interaction between the first molecule and the second molecule is dependent upon the presence of a particular structure on or within the first molecule; in other words the second molecule is recognizing and binding to a specific structure on or within the first molecule rather than to nucleic acids or to molecules in general. For example, if a second molecule is specific for structure “A” that is on or within a first molecule, the presence of a third nucleic acid sequence containing structure A will reduce the amount of the second molecule which is bound to the first molecule.

For example, the term “has the biological activity of a specifically named protein” when made in reference to the biological activity of a variant of the specifically named protein refers, for example, to a quantity of binding of an antibody that is specific for the specifically named protein to the variant which is preferably greater than 50% (preferably from 50% to 500%, more preferably from 50% to 200%, most preferably from 50% to 100%), as compared to the quantity of binding of the same antibody to the specifically named protein.

Reference herein to any specifically named nucleotide sequence includes within its scope fragments, homologs, and sequences that hybridize under stringent condition to the specifically named nucleotide sequence. The term “homolog” of a specifically named nucleotide sequence refers to an oligonucleotide sequence which exhibits greater than or equal to 50% identity to the sequence of interest. Alternatively, or in addition, a homolog of any specifically named nucleotide sequence is defined as an oligonucleotide sequence which has at least 95% identity with the sequence of the nucleotide sequence in issue. In another embodiment, the sequence of the homolog has at least 90% identity, and preferably at least 85% identity with the sequence of the nucleotide sequence in issue.

Exons, introns, genes and entire gene-sets are characteristically locatable with respect to one another. That is, they have generally invariant “genomic loci” or “genomic positions.” Genes distributed across one or several chromosomes can be mapped to specific locations on specific chromosomes. The field of “cytogenetics” addresses several aspects of gene mapping. First, optical microscopy reveals features of chromosomes that are useful as addresses for genes. In humans, chromosomes are morphologically distinguishable from one another and each (except for the Y-chromosome) has two distinct arms separated by a “centromere.” Each arm has distinctive “bands” occupied by specific genes. Disease-related changes in chromosome number, and changes in banding form the basis for diagnosing a number of diseases. “Microdissection” of chromosomes and DNA analysis of the microdissected fragments have connected specific DNA sequences to specific locations on chromosomes. In cancer, a region of a chromosome may duplicate or amplify itself or drop out entirely. FISH, mentioned above, and “comparative genomic hybridization” (“CGH”) have extended the reach of cytogenetic analysis to the extent of measuring genome alterations within and between individuals. CGH, for example, in which chromosomes from a normal cell are hybridized with a corresponding preparation from a cancer cell provides a means of directly determining cancer-related differences in copy number of chromosomal regions.

“Targeted therapeutics” is used herein to denote any therapeutic modality that affects only or primarily only the cells or tissues selected (“targeted”) for treatment. A monoclonal antibody specific for an antigen expressed only by a target (if retained by the target) is highly useful in targeted therapeutics. In the case of unwanted cells such as cancer cells, if the antibody doesn't induce destruction of the target directly, it may do so indirectly by carrying to the target, for example, a agent coupled to the antibody. On the other hand, agents that suppress processes that tend to promote uncontrolled proliferation of cells (“antineoplastic agents”) can be delivered to target sites in this manner.

The term “agent” is used herein in its broadest sense to refer to a composition of matter, a process or procedure, a device or apparatus employed to exert a particular effect. By way of non-limiting example, a surgical instrument may be employed by a practitioner as an “excising” agent to remove tissue from a subject; a chemical may be used as a pharmaceutical agent to remove, damage or neutralize the function of a tissue, etc. Such pharmaceutical agents are said to be “anticellular.” Cells may be removed by an agent that promotes apoptosis. A variety of toxic agents, including other cells (e.g., cytotoxic T-cell lymphocytes) and their secretions, and a plethora of chemical species, can damage cells.

The term “by-stander”, as used herein, refers to a process or event initiated or affected by another, causative event or process

The term “knockdown”, as used herein, refers to a method of selectively preventing the expression of a gene in an individual.

The term “oncogene”, as used herein, refers to any gene that regulates a process affecting the suppression of abnormal proliferative events.

The term “single nucleotide polymorphism” or “SNP”, as used herein, refers to a DNA sequence variation occurring when a single nucleotide in the genome (or other shared sequence) differs between members of a species or between paired chromosomes in an individual. Single nucleotide polymorphisms may fall within coding sequences of genes, non-coding regions of genes, or in the intergenic regions between genes. Single nucleotide polymorphisms within a coding sequence will not necessarily change the amino acid sequence of the protein that is produced, due to degeneracy of the genetic code. A Single nucleotide polymorphism in which both forms lead to the same polypeptide sequence is termed synonymous (sometimes called a silent mutation)—if a different polypeptide sequence is produced they are non-synonymous. Single nucleotide polymorphisms that are not in protein-coding regions may still have consequences for gene splicing, transcription factor binding, or the sequence of non-coding RNA.

The term “tissue array” or “tissue microarray”, as used herein, refers to high throughput platforms for the rapid analysis of protein, RNA, or DNA molecules. These arrays can be used to validate the clinical relevance of potential biological targets in the development of diagnostics, therapeutics and to study new disease markers and genes. Tissue arrays are suitable for genomics-based diagnostic and drug target discovery.

As used herein, the term “shRNA” or “short hairpin RNA” refers to a sequence of ribonucleotides comprising a single-stranded RNA polymer that makes a tight hairpin turn on itself to provide a “double-stranded” or duplexed region. shRNA can be used to silence gene expression via RNA interference. shRNA hairpin is cleaved into short interfering RNAs (siRNA) by the cellular machinery and then bound to the RNA-induced silencing complex (RISC). It is believed that the complex inhibits RNA as a consequence of the complexed siRNA hybridizing to and cleaving RNAs that match the siRNA that is bound thereto.

As used herein, the term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi inhibits the gene by compromising the function of a target RNA, completely or partially. Both plants and animals mediate RNAi by the RNA-induced silencing complex (RISC); a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs. Carthew has reported (Curr. Opin. Cell Biol. 13(2): 244-248 (2001)) that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

As used herein, the term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand”; the strand homologous to the target RNA molecule is the “sense strand”, and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “xenograft”, as used herein, refers to the transfer or transplant of a cell(s) or tissue from one species to an unlike species (or genus or family).

The term “orthotopic” or “orthotopic xenograft”, as used herein, refers to a cell or tissue transplant grafted into its normal place in the body.

The term “fluorescent activated cell sorting” or “FACS”, as used herein, refers to a technique for counting, examining, and sorting microscopic particles suspended in a stream of fluid. It allows simultaneous multiparametric analysis of the physical and/or chemical characteristics of single cells flowing through an optical and/or electronic detection apparatus. Generally, a beam of light (usually laser light) of a single wavelength is directed onto a hydro-dynamically focused stream of fluid. A number of detectors are aimed at the point where the stream passes through the light beam; one in line with the light beam (Forward Scatter, correlates to cell volume) and several perpendicular to the beam, (Side Scatter, correlates to the inner complexity of the particle and/or surface roughness) and one or more fluorescent detectors. Each suspended particle passing through the beam scatters the light in some way, and fluorescent chemicals found in the particle or attached to the particle may be excited into emitting light at a lower frequency than the light source. By analyzing the combinations of scattered and fluorescent light picked up by the detectors it is then possible to derive information about the physical and chemical structure of each individual particle.

The term “data mining”, as used herein, refers to the automated or convenient extraction of patterns representing knowledge implicitly stored or captured in large databases, data warehouses, internet websites, other massive information repositories, or data streams.

The terms “overexpress”, “overexpressing” and grammatical equivalents, as used herein, refer to the production of a gene product at levels that exceed production in normal or control cells. The term “overexpression” or “highly expressed” may be specifically used in reference to levels of mRNA to indicate a higher level of expression than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed, the amount of 28S rRNA (an abundant RNA transcript present at essentially the same amount in all tissues) present in each sample can be used as a means of normalizing or standardizing the mRNA-specific signal observed on Northern blots. Overexpression may likewise result in elevated levels of proteins encoded by said mRNAs.

The term “heatmap”, as used herein, refers to a graphical representation of data where the values obtained from a variable two-dimensional map are represented as colors. As related to the field of molecular biology, heat maps typically represent the level of expression of multiple genes across a number of comparable samples as obtained from a microarray.

The term “phage display”, as used herein, refers to the integration/ligation of numerous genetic sequences from a DNA library, consisting of all coding sequences of a cell, tissue or organism library into the genome of a bacteriophage (i.e. phage) for high-throughput screening protein-protein and/or protein-DNA interactions. Using a multiple cloning site, these fragments are inserted in all three possible reading frames to ensure that the cDNA is translated. DNA fragments are then expressed on the surface of the phage particle as part of it coat protein. The phage gene and insert DNA hybrid is then amplified by transforming bacterial cells (such as TG1 E. coli cells), to produce progeny phages that display the relevant protein fragment as part of their outer coat. By immobilizing relevant DNA or protein target(s) to the surface of a well, a phage that displays a protein that binds to one of those targets on its surface will remain while others are removed by washing. Those that remain can be eluted, used to produce more phage (by bacterial infection with helper phage) and so produce an enriched phage mixture. Phage eluted in the final step can be used to infect a suitable bacterial host, from which the phagemids can be collected and the relevant DNA sequence excised and sequenced to identify the relevant, interacting proteins or protein fragments.

The term “apoptosis”, as used herein, refers to a form of programmed cell death in multicellular organisms that involves a series of biochemical events that lead to a variety of morphological changes, including blebbing, changes to the cell membrane such as loss of membrane asymmetry and attachment, cell shrinkage, nuclear fragmentation, chromatin condensation, and chromosomal DNA fragmentation. Defective apoptotic processes have been implicated in an extensive variety of diseases; for example, defects in the apoptotic pathway have been implicated in diseases associated with uncontrolled cell proliferations, such as cancer.

The term “bioluminescence imaging” or “BLI”, as used herein, refers to the noninvasive study of ongoing biological processes in living organisms (for example laboratory animals) using bioluminescence, the process of light emission in living organisms. Bioluminescence imaging utilizes native light emission from one of several organisms which bioluminescence. The three main sources are the North American firefly, the sea pansy (and related marine organisms), and bacteria like Photorhabdus luminescens and Vibrio fischeri. The DNA encoding the luminescent protein is incorporated into the laboratory animal either via a virus or by creating a transgenic animal. While the total amount of light emitted via bioluminescence is typically small and not detected by the human eye, an ultra-sensitive CCD camera can image bioluminescence from an external vantage point. Common applications of BLI include in vivo studies of infection (with bioluminescent pathogens), cancer progression (using a bioluminescent cancer cell line), and reconstitution kinetics (using bioluminescent stem cells).

The term “consensus region” or “consensus sequence”, as used herein, refers to the conserved sequence motifs that show which nucleotide residues are conserved and which nucleotide residues are variable when comparing multiple DNA, RNA, or amino acid sequence alignments. When comparing the results of a multiple sequence alignment, where related sequences are compared to each other, and similar functional sequence motifs are found. The consensus sequence shows which residues are conserved (are always the same), and which residues are variable. A consensus sequence may be a short sequence of nucleotides, which is found several times in the genome and is thought to play the same role in its different locations. For example, many transcription factors recognize particular consensus sequences in the promoters of the genes they regulate. In the same way restriction enzymes usually have palindromic consensus sequences, usually corresponding to the site where they cut the DNA. Splice sites (sequences immediately surrounding the exon-intron boundaries) can also be considered as consensus sequences. In one aspect, a consensus sequence defines a putative DNA recognition site, obtained for example, by aligning all known examples of a certain recognition site and defined as the idealized sequence that represents the predominant base at each position. Related sites should not differ from the consensus sequence by more than a few substitutions.

The term “linkage”, or “genetic linkage,” as used herein, refers to the phenomenon that particular genetic loci of genes are inherited jointly. The “linkage strength” refers to the probability of two genetic loci being inherited jointly. As the distance between genetic loci increases, the loci are more likely to be separated during inheritance, and thus linkage strength is weaker.

The term “neighborhood score”, as used herein, refers to the relative value assigned to a genomic locus based on a geometry-weighted sum of expression scores of all the genes on a given chromosome, as a measurement of the copy number status of the locus. A positive neighborhood score is indicative of an increase in copy number, whereas a negative neighborhood score is indicative of a decrease in copy number.

The term “expression score”, as used herein, refers to the expression differences (i.e., the level of transcription (RNA) or translation (protein)) between comparison groups on a given chromosome. The expression score for a given gene is calculated by correlating the level of expression of said gene with a phenotype in comparison. For example, an expression score may represent a comparison of the expression differences of a given gene in normal vs. abnormal conditions, such as parental vs. drug-resistant cell lines. As used herein, the term “regional expression score” refers to the expression score of gene(s) in proximity to the locus in consideration. Since linkage strength between genetic loci decreases (i.e. decays) as the distance between them increases, the “regional expression score” more accurately reflects the expression differences between comparison groups by assigning greater weight to the expression scores of genes in proximity to the locus in consideration.

The terms “geometry-weighted” or “geometry-weighted sum”, as used herein, refers to the significance attached to a given value, for example an “expression score”, based on physical position, including but not limited to genomic position. Since linkage strength between genetic loci decreases (i.e. decays) as the distance between them increases, the “weight” assigned to a given value is adjusted accordingly.

The term “copy number alteration” or “CNA”, as used herein, refers to the increase (i.e. genomic gain) or decrease (i.e. genomic loss) in the number of copies of a gene at a specific locus of a chromosome as compared to the “normal” or “standard” number of copies of said gene that locus. As used herein, an increase in the number of copies of a given gene at a specific locus may also be referred to as an “amplification” or “genomic amplification” and should not be confused with the use of the term “amplification” as it relates, for example, to amplification of DNA or RNA in PCR and other experimental techniques.

The term “clonogenic assay”, as used herein, refers to a technique for studying whether a given cancer therapy (for example drugs or radiation) can reduce the clonogenic survival and proliferation of tumor cells. While any type of cell may be used, human tumor cells are commonly used for oncological research. The term “clonogenic” refers to the fact that these cells are clones of one another.

The term “adjuvant therapy”, as used herein, refers to additional treatment given after the primary treatment to increase the chances of a cure. In some instances, adjuvant therapy is administered after surgery where all detectable disease has been removed, but where there remains a statistical risk of relapse due to occult disease. If known disease is left behind following surgery, then further treatment is not technically “adjuvant”. Adjuvant therapy may include chemotherapy, radiation therapy, hormone therapy, or biological therapy. For example, radiotherapy or chemotherapy is commonly given as adjuvant treatment after surgery for a breast cancer. Oncologists use statistical evidence to assess the risk of disease relapse before deciding on the specific adjuvant therapy. The aim of adjuvant treatment is to improve disease-specific and overall survival. Because the treatment is essentially for a risk, rather than for provable disease, it is accepted that a proportion of patients who receive adjuvant therapy will already have been cured by their primary surgery. Adjuvant chemotherapy and radiotherapy are often given following surgery for many types of cancer, including colon cancer, lung cancer, pancreatic cancer, breast cancer, prostate cancer, and some gynecological cancers.

The term “matched samples”, as used herein, as for example “matched cancer samples” refers to a sample in which individual members of the sample are matched with every other sample by reference to a particular variable or quality other than the variable or quality immediately under investigation. Comparison of dissimilar groups based on specified characteristics is intended to reduce bias and the possible effects of other variables. Matching may be on an individual (matched pairs) or a group-wide basis.

The term “genomic segments”, as used herein, refers to any defined part or region of a chromosome, and may contain zero, one or more genes.

The term “co-administer”, as used herein, refers to the administration of two or more agents, drugs, and/or compounds together (i.e. at the same time).

The term “diagnose” or “diagnosis”, as used herein, refers to the determination, recognition, or identification of the nature, cause, or manifestation of a condition based on signs, symptoms, and/or laboratory findings.

### DETAILED DESCRIPTION

Applicants' RNA interference-based analysis of the aforementioned 150 genes in breast cancer cells has identified transcriptional regulators of fat synthesis and storage as being essential for the survival of these cells. These transcription factors include NR1D1 (RevErba), and the PPARγ binding protein (PBP). Both genes reside on ERBB2-containing 17q12-21 amplicons^{6,7} and coordinately upregulate the de novo fatty acid synthesis pathway that is highly active in these cells. Applicants' results demonstrate that the cells of this aggressive form of breast cancer are genetically preprogrammed to depend on fatty acid synthesis for energy production necessary for survival. Accordingly, in various embodiments, the invention provides methods of identifying agents that reduce or prevent the proliferation of cancer cells, or kill them.

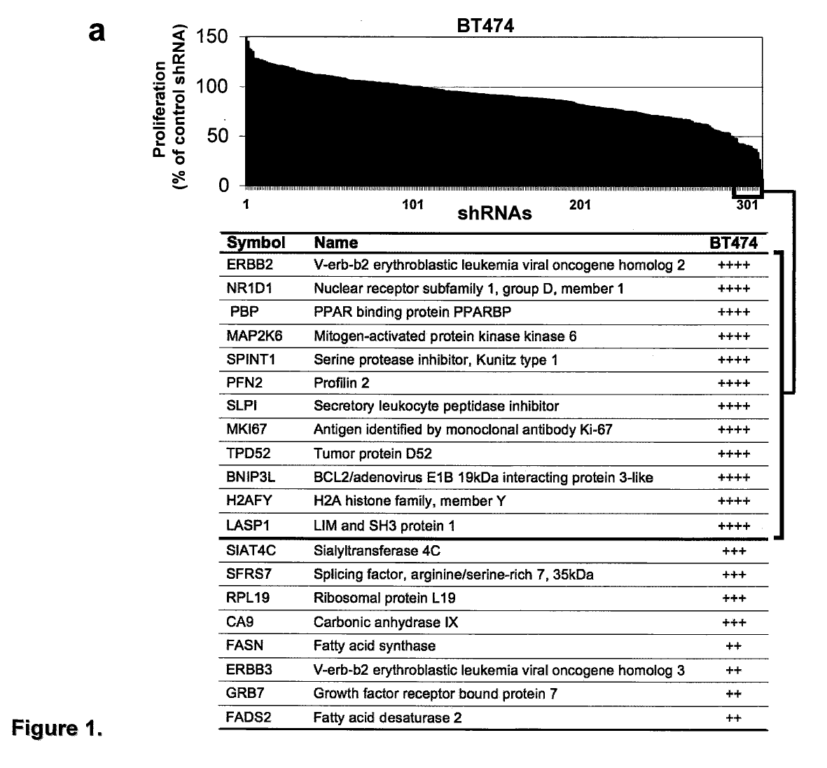

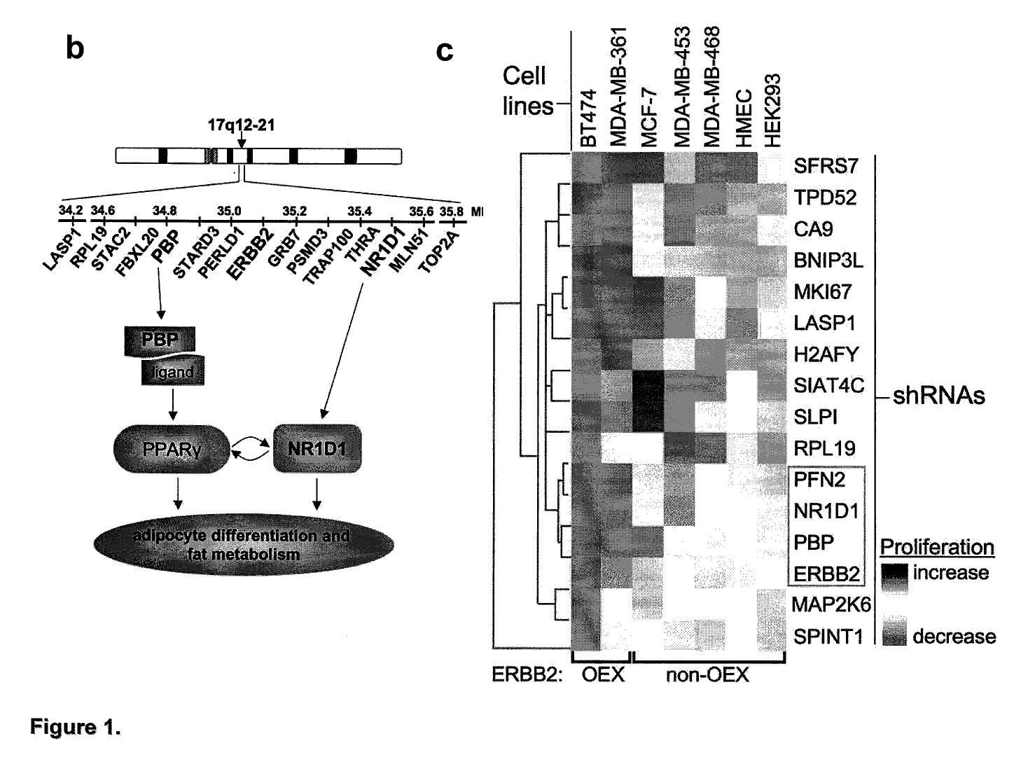

Amplification of the ERBB2 oncogene occurs in 10-34% of breast cancer cases and is correlated with aggressive disease and poor clinical outcome¹. Herceptin, a monoclonal antibody targeting the extracellular domain of ERBB2, has been widely hailed as the first “next generation” cancer therapy. However, its success has been tempered by response rates of only 30% when used as a single agent therapy in patients with metastatic ERBB2-positive breast cancer². A large number of genes co-overexpressed with ERBB2 in this tumour type have been proposed to contribute to the aggressiveness of this cancer³⁻⁵. Applicants have performed an unbiased functional RNAi screen⁸⁻¹⁰ to determine if any of these co-overexpressed genes are essential for ERBB2 positive breast cancer cell survival. 309 short-hairpin RNAs (shRNAs)⁸ were used individually to target 141 of these genes in the ERBB2-positive BT474 breast cancer cell line and effects on cells were monitored using alamarBlue, a fluorimetric indicator of both cell proliferation and viability¹¹. shRNAs targeting eleven distinct genes resulted in more than a 50% decrease in proliferation (FIG. 1a), including, as expected, the ERBB2 shRNA. Other positive shRNAs with significant effects on proliferation silence genes with previously reported roles in cancer, such as KI67, TPD52 and CA9 (FIG. 1a) validating the approach.

Surprisingly, three of the shRNAs that had the most dramatic effect on cell proliferation targeted genes that had been shown previously to play roles in adipogenesis. This process involves the synthesis of large quantities of fatty acids and their storage within cells as neutral fats, mainly triglycerides. The shRNA targets included genes for the nuclear receptor subfamily 1, group D, member 1 (NR1D1; RevErba)¹², the PPARγ binding protein (PBP)¹³ and the MAP kinase, MAP2K6¹⁴ (FIG. 1a). Two others, fatty acid synthase (FASN) and fatty acid desaturase 2 (FADS2), which, when silenced, had a moderate effect on cell proliferation, are also involved in lipid metabolism. Of these, NR1D1 and PBP are particularly interesting. They are tightly linked to ERBB2 on chromosome 17 and frequently reside on the 17q12-21 amplicons found in these tumours^{6,7} (FIG. 1b). Several studies have shown that they are consistently co-overexpressed with ERBB2 in breast tumours^{15,16} and are among the six genes that comprise the ERBB2 gene expression signature seen in breast cancers⁵. These genes are also functionally linked. PBP is a co-activator of peroxisome proliferator activated receptor gamma (PPARγ)¹³. PPARγ positively regulates upwards of 30 genes related to adipogenesis¹⁷ and has been shown to activate NR1D1 transcription¹⁸. NR1D1 is a transcription factor that promotes adipocyte differentiation, although few downstream effectors are known¹². Previous studies suggest that it may inhibit expression of antiadipogenic genes, or potentiate the activity of PPARγ¹² (FIG. 1b). Recently, NR1D1 has been shown to bind heme as a requisite ligand for transcriptional suppression¹⁹ and has also been identified as a component of the circadian clock through its suppression of BMAL1 (Brain and Muscle Arnt-like protein 1) via recruitment of the N—CoR/histone deacetylase 3 corepressor²⁰.

To assess the specificity of effect of each shRNA on cell proliferation, those causing the greatest decrease in BT474 cell proliferation were transfected in a variety of cell lines, including ERBB2 overexpressing (BT474, MDA-MB-361) and nonoverexpressing breast cancer cell lines (MCF-7, MDA-MB-453, MDA-MB-468), normal human mammary epithelial cells (HMECs) and a non-breast tumorigenic cell line (HEK 293FT). Proliferation rates of shRNA-transfected cells were then measured for each shRNA-transfected cell line and the results were subjected to cluster analysis and displayed as a heat map and dendrogram (FIG. 1c). PBP, NRID1 and PFN2 shRNAs clustered with ERBB2. Each specifically decreased the viability of ERBB2-positive cells yet was without effect on other cell lines or HMECs (FIG. 1c).

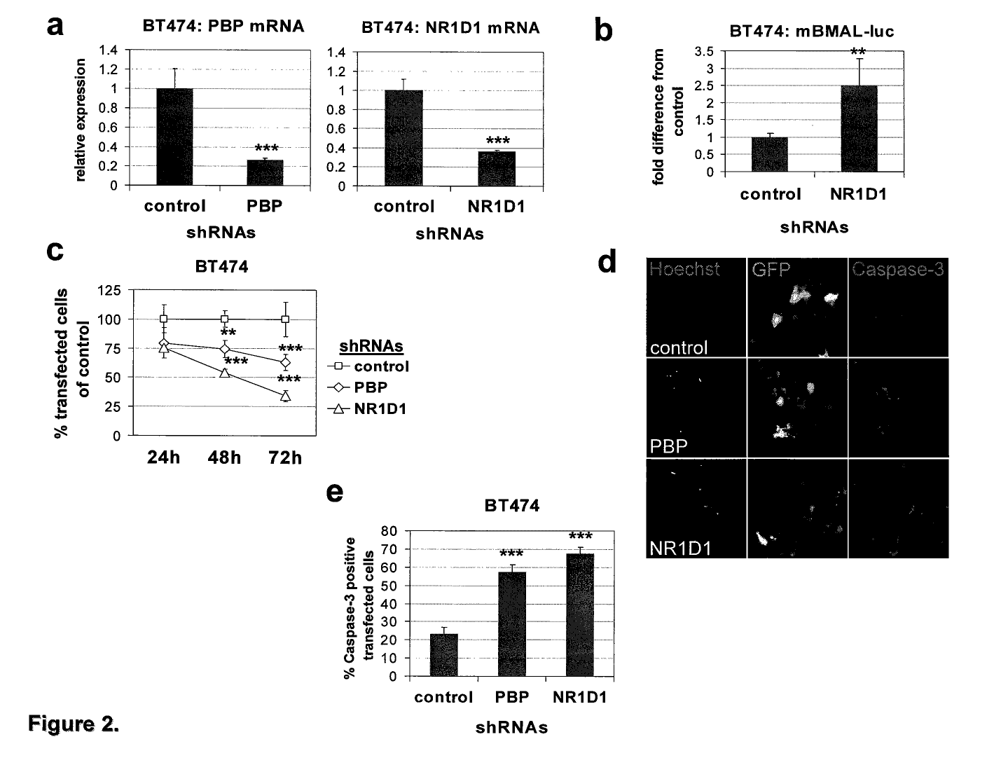

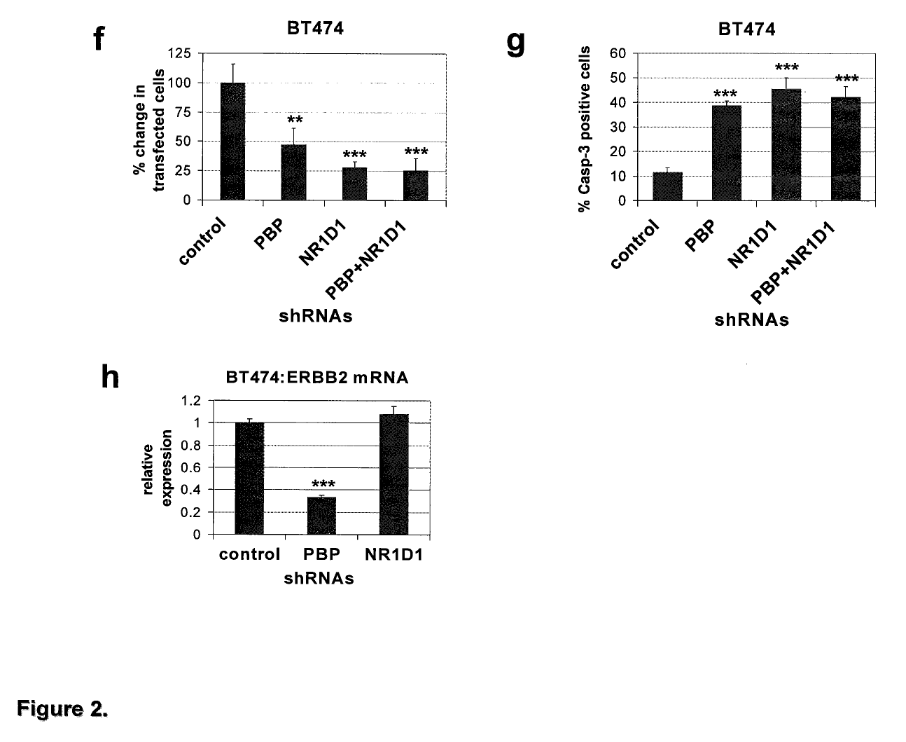

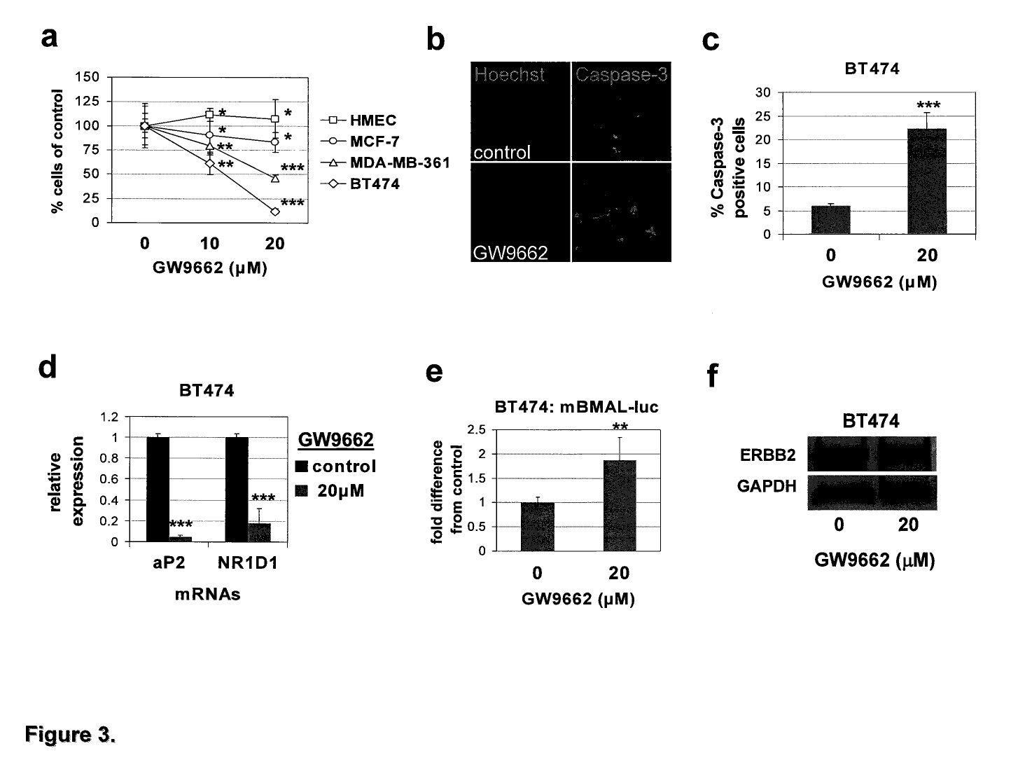

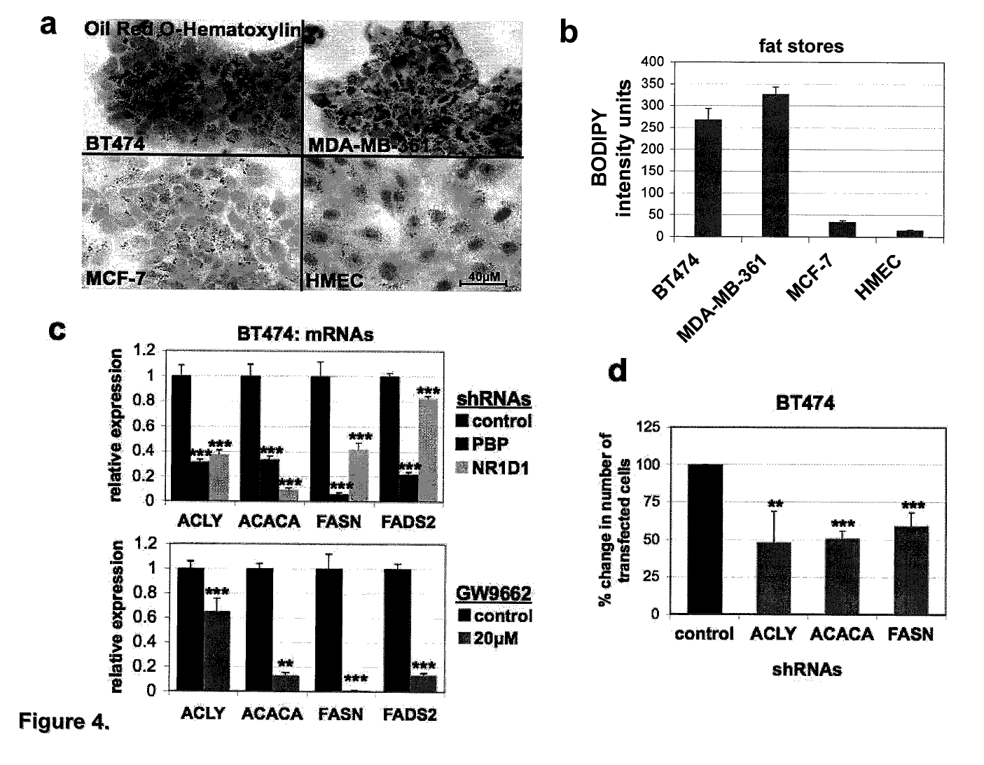

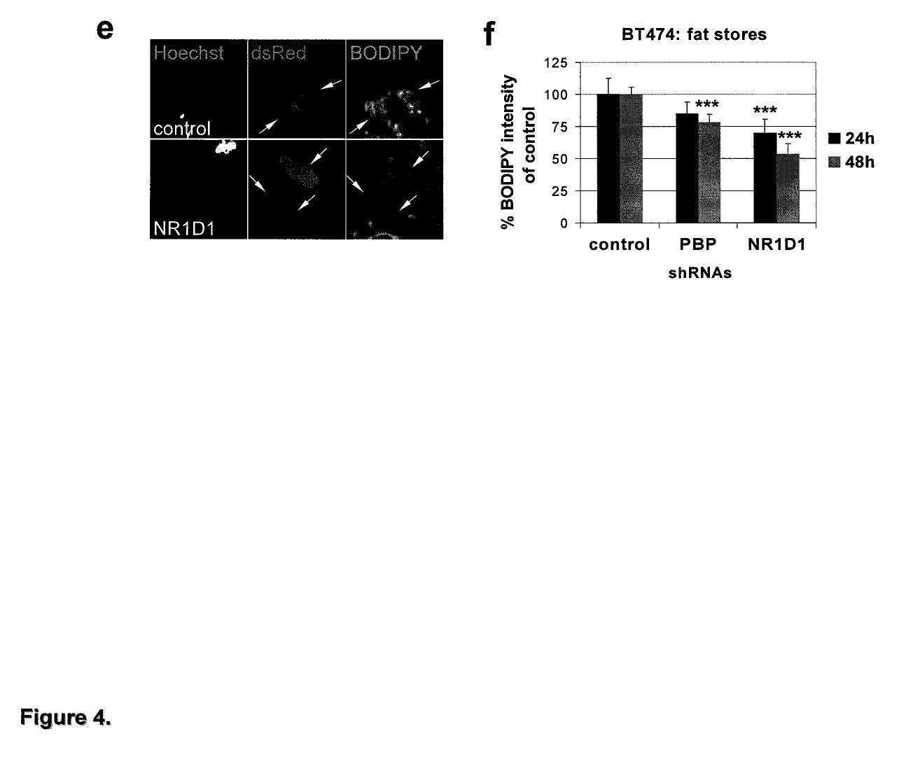

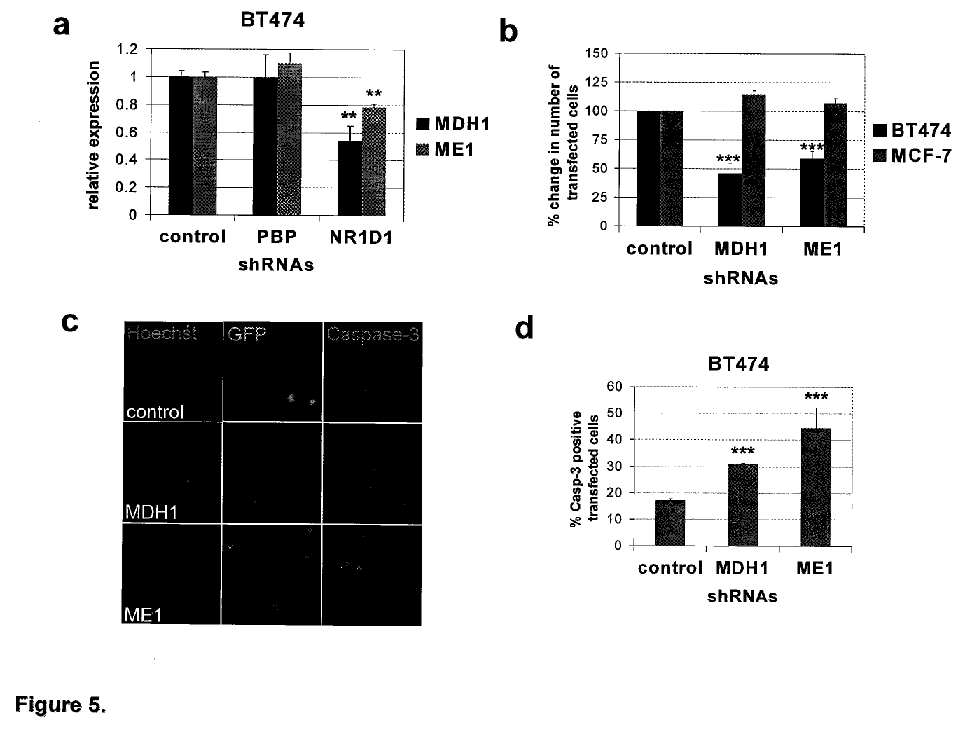

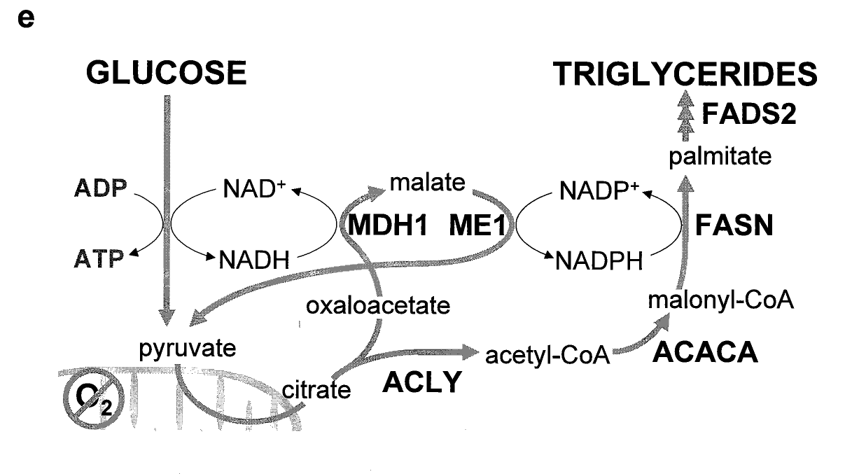

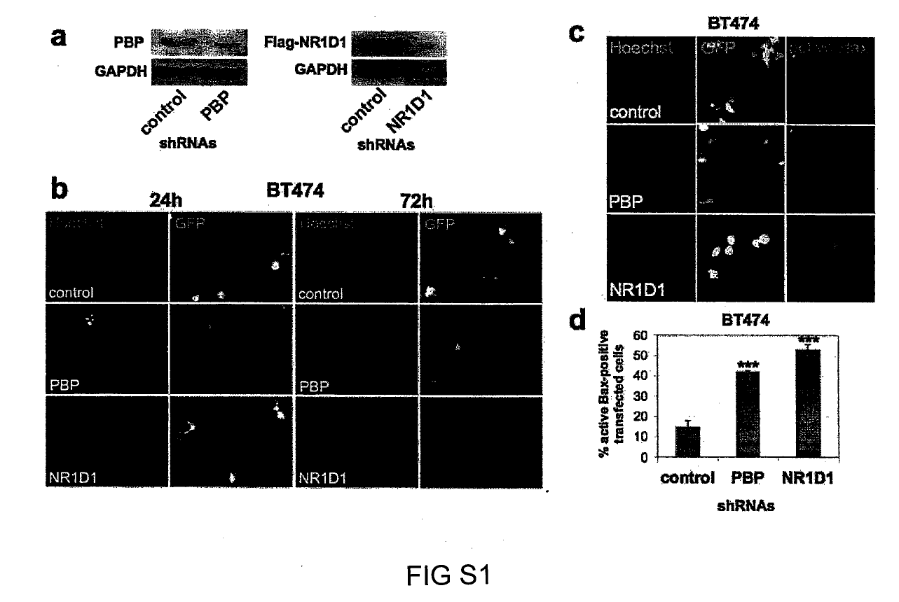

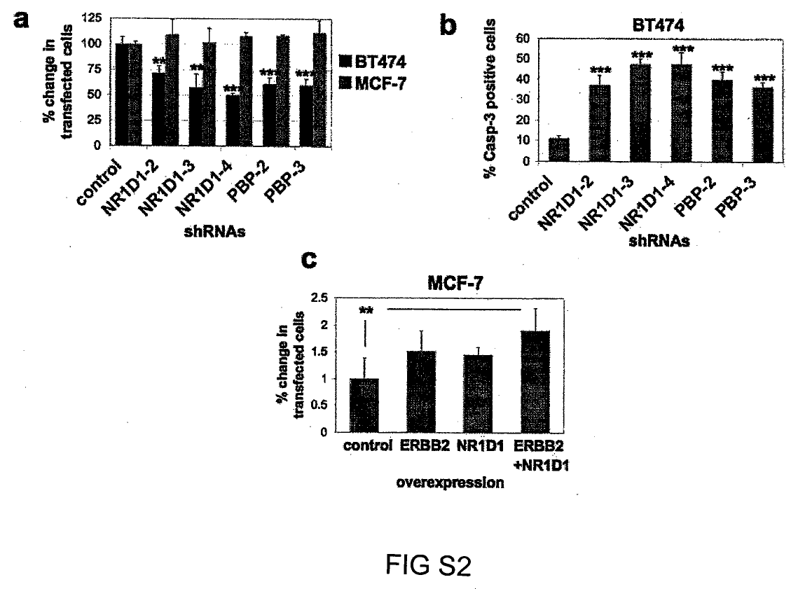

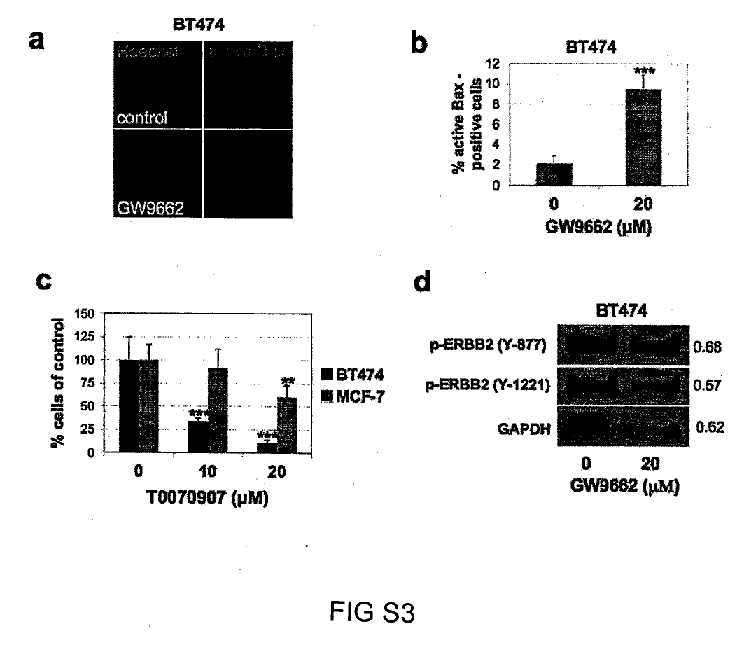

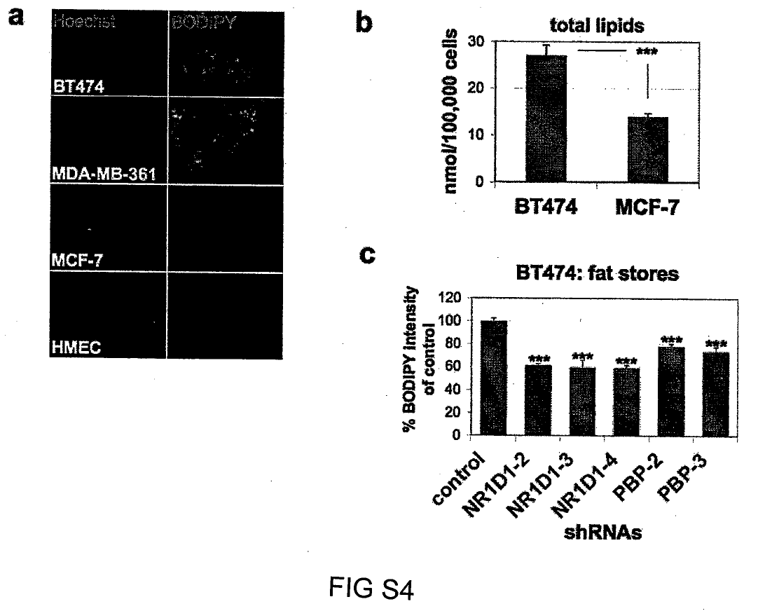

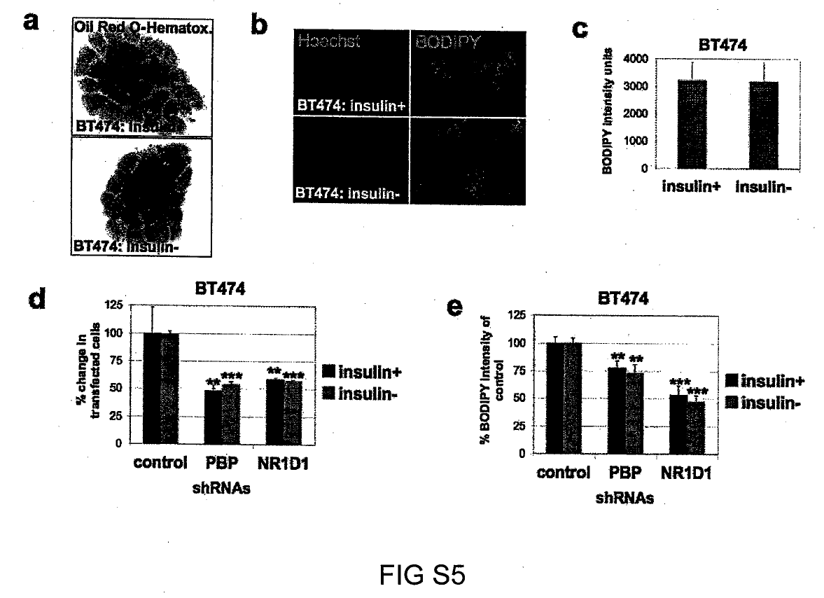

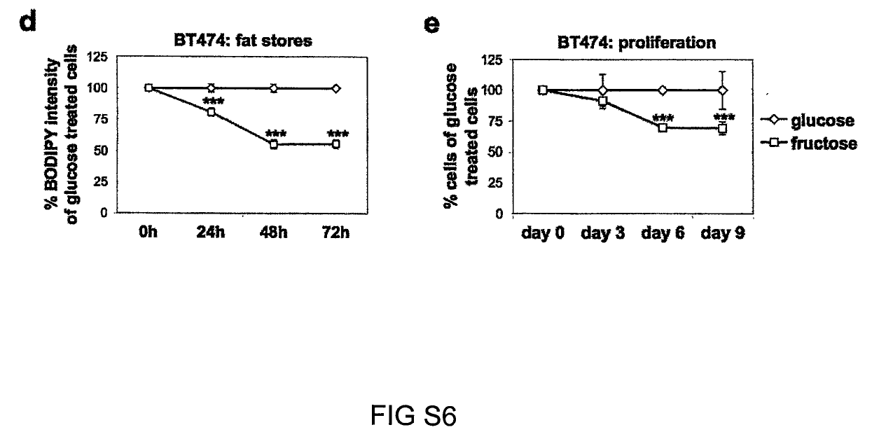

Transfection with NR1D1 and PBP shRNAs resulted in more than a 60% decrease in mRNA levels of their targets in BT474 cells (FIG. 2a), as well as in decreased protein levels (FIG. S1a). The NR1D1 shRNA also induced a 2.5-fold up-regulation of a BMAL1 reporter (FIG. 2b) consistent with reports of its activity in other cell types^{20,21}. Decreased NR1D1 and PBP expression resulted in cell death as indicated by the time-dependent decrease of cell number in populations transfected with NR1D1 and PBP shRNAs (FIG. 2c and FIG. S1b). Assays of Caspase-3 or Bax activation indicated that this was due to a 2.5 to 3-fold increase in apoptosis, which was initially observed within 48 h of shRNA transfection and resulted in extensive cell death after 72 h (FIG. 2c-e, and FIG. S1c, d). Three more shRNAs targeting NR1D1 and two targeting PBP also resulted in cell death and apoptosis specifically of BT474 cells (FIG. S2a, b).

The common roles of NR1D1 and PBP genes in regulating metabolism prompted us to investigate whether their effects on BT474 cells are through the same or different pathways. Simultaneous transfection of the NR1D1 shRNA with the PBP shRNA did not exacerbate the cell death (FIG. 2f) and apoptosis (FIG. 2g) observed when using the NR1D1 shRNA alone, indicating that the survival advantage that the two genes confer in these cells is via the same pathway. We also examined whether the actions of the two genes are through ERBB2. PBP knockdown significantly decreased the mRNA levels of ERBB2, however, NR1D1 inhibition had no effect on these levels (FIG. 2h). This indicates that the effects of the two genes on BT474 cells are ERBB2-independent, since the two genes act via the same pathway and ERBB2 is not a common downstream effector. This notion is further supported by the additive effect that was observed in the proliferation of the ERBB2-negative MCF-7 cells, when ERBB2 and NR1D1 were simultaneously overexpressed (FIG. S2c). The above results show that NR1D1 and PBP act in BT474 cells in a common pathway that does not involve ERBB2, indicating that their co-overexpression with ERBB2 in breast cancer is most likely due to their tight genetic linkage (FIG. 1b), rather than due to their functional association.

The functional relationships of NRID1 and PBP to PPARγ established in adipocytes^{13,18} suggest a role for PPARγ in the function of NR1D1 and PBP in ERBB2 positive breast cancer cells. To examine this, we treated cells with an antagonist (GW9662) that binds irreversibly to PPARγ, inhibiting its function as a transcriptional activator. Similar to the effects seen with NR1D1 and PBP shRNAs, ERBB2-positive cells were more sensitive to drug treatment than MCF-7 and HMEC cells (FIG. 3a). This treatment resulted in the apoptosis of BT474 cells (FIG. 3b, c and FIG. S3a, b). A second PPARγ antagonist produced the same effects (FIG. S3c). Inhibition of PPARγ activity by GW9662 was confirmed by decreased mRNA levels of aP2 (FABP4, fatty acid binding protein 4), a major PPARγ target gene¹⁷ (FIG. 3d). GW9662 also dramatically decreased NR1D1 message levels, which was consistent with previous observations¹⁸ (FIG. 3d) and resulted in increased BMAL1 reporter activity (FIG. 3e). Neither ERBB2 protein levels (FIG. 3f), nor ERBB2 phosphorylation (FIG. S3d) were significantly affected by GW9662 treatment, further demonstrating that the pathway that is activated by NR1D1, PBP and PPARγ does not involve ERBB2. Since PPARγ inhibitors have the same phenotypic effects and the same pattern of cell line-specificity as the NR1D1 and PBP shRNAs, we infer that ERBB2-positive breast cancer cells require the adipogenesis-promoting activity of PPARγ for survival.

The emergence of genes related to adipogenesis in the screen suggests that fat production and storage is critical to ERBB2-positive breast cancer cell survival. Stains of neutral fat, Oil-Red O and the fluorescent indicator BODIPY 493/503, both indicate that ERBB2-positive breast cancer cells have dramatically higher levels of triglyceride stores, compared to MCF-7 and HMEC cells (FIG. 4a, b and FIG. S4a). When quantified by mass spectrometry, differences were also observed in total cellular fats. Since we were primarily interested in learning why the ERBB2-positive cell line (BT474) was sensitive to treatment with shRNAs and PPARγ antagonists while the EBRB2-negative cell line (MCF-7) was relatively resistant, we focused on differences between these two lines. BT474 cells were also found to contain more than twice the amount of fatty acids of MCF-7 cells (FIG. S4b). These results are consistent with previous reports that documented an association between ERBB2 and fatty acid synthase (FASN) expression²².

The three major enzymes of de novo fatty acid synthesis, FASN, which was also identified in the screen (FIG. 1a), ATP citrate lyase (ACLY) and acetyl-coenzyme A carboxylase alpha (ACACA) were examined as potential downstream targets of NR1D1 and PBP. The transcript level of each enzyme was reduced after transfection with NR1D1 and PBP shRNAs or PPARγ antagonist treatment (FIG. 4c). In agreement with previous studies on FASN, ACLY and ACACA, which establish their relevance to tumorigenesis^{6,22-24}, shRNAs targeting FASN, ACLY and ACACA significantly decreased BT474 cell viability (FIG. 4d). FADS2, which encodes a desaturase that converts saturated fatty acids, like palmitate, to less toxic unsaturated fatty acids prior to incorporation into triglycerides was also identified in our screen (FIG. 1a). FADS2 transcript levels were also downregulated after NR1D1-PBP knockdown or PPARγ inhibition (FIG. 4c). These results show that NR1D1, PBP and PPARγ coordinate the expression of the FASN, ACLY, ACACA and FADS2 genes in ERBB2-positive breast cancer cells, likely maximizing the fatty acid synthetic capacity of these cells.

The importance of fatty acid synthesis for the survival of ERBB2-positive cancer cells is not due to the level of stored fats but rather the synthetic process itself. Targeting NR1D1 and PBP with shRNAs significantly decreased fat stores in BT474 cells by 47% and 30% (FIG. 4e, f and FIG. S4c), respectively, in the first 48 h after transfection, and before extensive cell death occurred. Neither fat accumulation, nor the effects of NRID1 and PBP shRNAs on decreasing cell viability and fat storage were altered by supplementation of the BT474 medium with insulin (FIG. S5). Similar decreases in fat stores were observed in BT474 cells grown in media containing the alternative fuel sources galactose or fructose (FIG. S6a-d). Importantly, these decreases did not lead to cell death (FIG. S6e). These results indicate that the survival function provided by NR1D1 and PBP is due to increased activity of the fatty acid synthesis pathway and not the increased levels of the products of this pathway that are found in ERBB2-positive breast cancer cells.

ERBB2-positive breast cancer cells store fatty acids at 10 times the level of other breast cancer cells. A rationale for fat storage as a survival mechanism has been suggested by studies focused on FASN which have shown that increased fatty acid synthesis in some cancer cells is a feature of aerobic glycolysis^{25,26}, the altered tumour cell energy metabolism first proposed by Warburg²⁷. Oxygen does not serve as the terminal electron acceptor in cells with this physiology. To avoid low NAD⁺/NADH ratios that would eventually feed back to inhibit glycolysis, electrons are incorporated into other molecules such as lactate with the concomitant regeneration of NAD⁺. Since the reaction catalyzed by FASN uses NADPH as a cofactor, it is thought to play a role in indirectly regenerating NAD under these conditions. The transcriptional regulation by PBP of FASN as well as other fatty acid synthesis and storage enzymes whose concerted action is required for clearing electrons produced during glycolysis provides an explanation for the essential nature of these genes. This also predicts that other enzymes that might couple the transfer of electrons from glycolysis to fat synthesis would also be required for ERBB2-positive breast cancer cell survival. Two such enzymes are cytosolic malate dehydrogenase 1 (MDH1), which is the key enzyme in the production of cytoplasmic malate and normally functions in the disposition of cytoplasmic electrons via the malate-aspartate shuttle²⁸ and in pyruvate-citrate cycling²⁹, and malic enzyme 1 (ME1), which converts malate to pyruvate and is the primary source of NADPH required by FASN for palmitate synthesis²⁸ (FIG. 5e). Message levels of the MDH1 and ME1 enzymes after NR1D1 shRNA treatment were significantly decreased (FIG. 5a). In addition, targeting MDH1 and ME1 with RNAi, resulted in cell death (FIG. 5b) and apoptosis (FIG. 5c, d) of BT474 cells, showing that they are required specifically by ERBB2-positive breast cancer cells for survival. Taken together, these results support the notion that coordinate increases in the enzymes of the fatty acid synthesis pathway are essential for ERBB2-positive breast cancer cells because the end product of this pathway, palmitate, and its storage as non-toxic triglycerides serves as a sink for electrons, which allows for regeneration of NAD and continued glycolysis.

This study indicates that the tight genetic linkage between NR1D1, PBP and ERBB2 causes co-overexpression of these gene products such that ERBB2-positive breast cancer cells are genetically preprogrammed to depend on fatty acid synthesis for energy production and survival. In these cells, the major effect of inhibiting these genes is the acute inhibition of de novo fatty acid synthesis, which in turn impedes energy metabolism and triggers apoptosis. Although these effects are largely independent of ERBB2, further research is required to resolve its involvement, since ERBB2 has been shown in some studies to regulate FASN expression via the PI3K pathway and in others to be downregulated by FASN inhibition via PEA322. In any event, that inhibition of NR1D1 and PBP specifically increases the sensitivity of ERBB2-overexpressing cells to apoptosis indicates that they are excellent therapeutic targets for these tumors.

### Experimental

Cell culture and chemicals. Breast cancer cell lines BT474, MCF-7, MDA-MB-361, MDA-MB-453, MDA-MB-468 were obtained from ATCC. Human mammary epithelial cells (HMEC) were obtained from Cambrex. HEK 293FT cells were obtained from Invitrogen. BT474, MCF-7 and HEK 293FT cells were cultured in DMEM (Hyclone) supplemented with 10% FBS (Hyclone) and 100 U/μl of penicillin-streptomycin (Cellgro); BT474 medium was also supplemented with ITS (insulin, transferrin and selenium; Cellgro). MDA-MB-361 were cultured in RPMI-1640 (Hyclone) supplemented with 20% FBS and 100 U/μl of penicillin-streptomycin. MDA-MB-453 and MDA-MB-468 cells were cultured in Liebovitz L-15 medium (Hyclone) supplemented with 10% FBS and 100 U/μl of penicillin-streptomycin. HMECs were cultured in MEGM medium (Cambrex). The PPARγ antagonists GW9662 and T0070907 were obtained from Sigma-Aldrich.

RNAi screen, transfections and constructs. The flag-RevErba (flag-NR1D1) overexpression construct was a generous gift of M. Lazar. The LSXN-neu* (ERBB2) overexpression construct was a generous gift of L. Petti. shRNA constructs were expressed from the pSHAG-MAGIC 2 (pSM2) vector and derived from a genome-wide shRNA library³¹. ShRNAs targeting the firefly (Photinus pyralis) luciferase gene were used as controls. Transfection efficiency was monitored by co-transfection with a modified MSCV-Puro vector expressing green fluorescent protein (GFP). The alamarBlue (Biosource) assay was performed 96 h post-transfection, since BT474 cells have a population doubling time of ˜100 hours. For mature sequences of the shRNAs that produced the best results on decreasing BT474 viability, Table S1; a complete list is available in the RNAi Codex web page (http://codex.cshl.edu). The shRNAs targeting the luciferase gene were constructed as described in the RNAi Codex web page (http://codex.cshl.edu/scripts/newmain.pl) using a modified pSM2 vector containing the PheS gene (pSM2-PheS) in the cloning site, as a negative selection marker. The empty vector control (pSM2e) was constructed by XhoI and EcoRI (New England Biolabs) digestion of pSM2-PheS, followed by nucleotide filling of protruding ends by Klenow polymerase and blunt-end ligation. Transfections were performed using FuGENE 6 and HD FuGENE (Roche) according to the manufacturer's protocol. High-throughput transfections were performed using an EpMotion 5070 fluidics station (Eppendorf). For quantification of alamarBlue we used a BioTek HT Synergy plate reader. AlamarBlue values from the RNAi screens on other cell types were also calculated as above and subjected to hierarchical cluster analysis using Cluster 2.11 (M. Eisen Lab).

Cell viability—apoptosis assays. Live cell counts were performed using a hemocytometer after cell trypsinization and trypan blue staining. For high-throughput experiments, cells grown on 96-well plates were washed once with 1×PBS, fixed with 2.5% formaldehyde and stained with Hoechst 33342 (Molecular Probes-Invitrogen). Pictures of cells were acquired using an In Cell Analyzer 1000 (GE Healthcare) high-content imaging system, with a 20× objective. At least 30 fields were imaged per single experiment. Cell counts and statistics were then performed using the In Cell Investigator 3.4 high-content image analysis software (GE Healthcare). Apoptosis was detected by cleaved Caspase-3 and activated Bax immunofluoresence after 48 h of shRNA or GW9662 treatments. In this case, cells were fixed after treatment with 2.5% formaldehyde, washed with 1×PBS, permeabilized with 0.1% Triton-X 100 (Fisher Chemicals), blocked with 3% normal goat serum (Sigma-Aldrich), incubated with a 1:501:200 dilution of the primary antibody, washed with 1×PBS, incubated with a 1:800 dilution of the secondary antibody, washed again with 1×PBS and finally stained with Hoechst 33342 (Molecular Probes-Invitrogen). Cells were imaged by the In Cell Analyzer 1000 (GE Healthcare) or by a Leica TCS SP5 confocal microscope system (Leica Microsystems). At least 500 cells were counted for cleaved Caspase-3 or active Bax signal. Antibodies used: cleaved Caspase-3 (Asp175, #9661; Cell Signaling Technology), Bax (6A7; BD, San Jose, Calif.), Alexa Fluor 568 goat anti-rabbit IgG (#A11011; Invitrogen) and Alexa Fluor 568 goat anti-mouse IgG (#A-11004; Invitrogen). Immunoblotting. Cell extracts for western blots were obtained using RIPA buffer (1% Triton X-100, 40mM NaCl, 0.1% SDS, 10 mM Tris pH 8.0) supplemented with complete cocktail of proteinase inhibitors (Roche). For detection of phoshporylated epitopes, the PhosSTOP cocktail of phospatase inhibitors (Roche) was added in the lysis buffer. Protein extracts were separated by SDS-PAGE, transferred to Immobilon-P (Millipore) membranes and immunoblotted according to standard protocols. Blots were imaged using a FluorChem HD (Alpha Innotech) imaging system and images were analyzed by densitometry using the FluorChem 9900 software (Alpha Innotech). Antibodies used: anti-flag (M2; Stratagene), PBP (TRAP220, C-19; Santa Cruz), GAPDH (V-18; Santa Cruz Biotechnology), ERBB2 (clone 42; BD), phospho-ERBB2 (Tyr877, #2241; Cell Signaling Technology), phospho-ERBB2 (Tyr1221/1222, 6B12; Cell Signaling Technology), anti-rabbit IgG-HRP (sc-2204, Santa Cruz Biotechnology), anti-goat IgG-HRP (sc-2768, Santa Cruz Biotechnology), anti-mouse IgG-HRP (#31430; Pierce Biotechnology) qRT-PCR. For all qRT-PCR reactions, cells were harvested 48 h post-transfection. Due to low shRNA transfection efficiency of BT474 cells, cells were co-transfected with GFP and GFP positive cells were sorted prior to RNA extraction, using a BD FACSAria sorting system. Final population enrichment of GFP-positive cells was 70-75%. Total RNA was extracted from sorted cells using TRizol (Invitrogen) and cDNA was synthesized by reverse transcription of 2 μg of RNA in a 20 μl reaction using MMLV reverse transcriptase (Promega) at 42° C. for 1 h. RT-PCR reactions were performed using standard Taq polymerase (Fisher BioReagents). The primer pairs used were designed using ABI's Primer Express software (Table S2). After the initial denaturation step (95° C. for 3 min), PCR reactions consisted of 30-35 cycles of a 95° C.—15 step, a 52-55° C.—15 sec step and a 72° C.—20 sec step, followed by a final elongation step (72° C. for 5 min). PCR products were separated on 2% agarose—ethidium bromide gels. For quantitative determination of PCR product, a real-time reverse transcription PCR (RT-PCR) was performed on ABI PRISM 7900HT Sequence Detection System (Applied Biosystems, Foster City), using SYBR Green PCR Master Mix (Applied Biosystems). Primer pairs were the same as the ones used in regular RT-PCR. PCR reaction was run using standard conditions. After initial incubation at 95° C. for 2½ min, the amplification protocol consisted of 40 cycles of a 95° C.—15 sec step and a 60° C.—60 sec step. Product levels were calculated after normalization with β-actin control.

Reporter assays. The mBMAL1-luc reporter was a generous gift of U. Schibler. The pRL vector expressing renilla (Renilla reniformis) luciferase was obtained from Promega. Cells were transfected with the reporter construct plus pRL to normalize for transfection efficiency and the shRNA of interest; empty pSM2 (pSM2e) was used as control. Cells were lysed 48 h post-transfection and firefly (reporter) and renilla luciferase activities were measured using the Dual-Luciferase Reporter Assay kit (Promega) on a BioTek Synergy HT reader. The ratio of Firefly to Renilla luciferase activity was calculated and presented as percentage of control.

Metabolic assays. For detection of neutral fat stores, cells were either stained with 60% Oil Red O (Fisher Chemicals) or with 10 μg/ml 4,4-difluoro-1,3,5,7,8-pentamethyl-4-bora3a, 4a-diaza-s-indacene (BODIPY 493/503; Molecular Probes). For Oil Red O staining, cells were grown on coverslips, fixed with 10% formaldehyde, washed with 1×PBS, washed twice with 60% isopropanol, stained with 60% Oil Red O (Fisher Chemicals) in isopropanol for 1 h, washed several times with tap water and finally with distilled water and counter-stained for nuclei with Mayer's Hematoxylin (Sigma-Aldrich). Stained cells were visualized using the 60× objective of an Arcturus Veritas (Molecular Devices Corporation) microdissection system. For BODIPY assays, cells were grown on 96-well plates, fixed with 2.5% formaldehyde, washed with 1×PBS, stained with 10 μg/ml BODIPY 493/503; Molecular Probes and counter-stained with Hoechst 33342 (Molecular Probes) for nuclei identification. Transfected cells were monitored by co-transfecting with the pDsRed-Monomer-N1 vector (Clontech). Cells were imaged and analyzed using the In Cell Analyzer 1000—In Cell Investigator 3.4 system (GE Biosciences), as described above. For total fatty acid detection and quantification, total cellular lipids were extracted according to a standard procedure previously described 32.

Statistics. The Student's two-tailed t-test was employed. Comparisons in each case refer to the respected controls, unless otherwise indicated.

### REFERENCES

1. Ross, J. S. & Fletcher, J. A. The HER-2/neu oncogene in breast cancer: prognostic factor, predictive factor, and target for therapy. Stem Cells 16, 413-428 (1998).
2. Vogel, C. L. et al. Efficacy and safety of trastuzumab as a single agent in first-line treatment of HER2-overexpressing metastatic breast cancer. J Clin Oncol 20, 719726 (2002).
3. Perou, C. M. et al. Molecular portraits of human breast tumours. Nature 406, 747-752 (2000).
4. Mackay, A. et al. cDNA microarray analysis of genes associated with ERBB2 (HER2/neu) overexpression in human mammary luminal epithelial cells. Oncogene 22, 2680-2688 (2003).
5. Bertucci, F. et al. Identification and validation of an ERBB2 gene expression signature in breast cancers. Oncogene 23, 2564-2575 (2004).
6. Chin, K. et al. Genomic and transcriptional aberrations linked to breast cancer pathophysiologies. Cancer Cell 10, 529-541 (2006).
7. Kauraniemi, P. & Kallioniemi, A. Activation of multiple cancer-associated genes at the ERBB2 amplicon in breast cancer. Endocr Relat Cancer 13, 39-49 (2006).
8. Paddison, P. J. et al. A resource for large-scale RNA-interference-based screens in mammals. Nature 428, 427-431 (2004).
9. Berns, K. et al. A functional genetic approach identifies the PI3K pathway as a major determinant of trastuzumab resistance in breast cancer. Cancer Cell 12, 395-402 (2007).
10. Silva, J. M. et al. Profiling essential genes in human mammary cells by multiplex RNAi screening. Science 319, 617-620 (2008).
11. Kourtidis, A., Eifert, C. & Conklin, D. S. RNAi applications in target validation. Ernst Schering Res Found Workshop, 1-21 (2007).
12. Laitinen, S., Fontaine, C., Fruchart, J. C. & Staels, B. The role of the orphan nuclear receptor Rev-Erb alpha in adipocyte differentiation and function. Biochimie 87, 21-25 (2005).
13. Zhu, Y., Qi, C., Jain, S., Rao, M. S. & Reddy, J. K. Isolation and characterization of PBP, a protein that interacts with peroxisome proliferator-activated receptor. J Biol Chem 272, 25500-25506 (1997).
14. Engelman, J. A. et al. Constitutively active mitogen-activated protein kinase kinase 6 (MKK6) or salicylate induces spontaneous 3T3-L1 adipogenesis. J Biol Chem 274, 35630-35638 (1999).
15. Zhu, Y. et al. Amplification and overexpression of peroxisome proliferator-activated receptor binding protein (PBP/PPARBP) gene in breast cancer. Proc Natl Acad Sci USA 96, 10848-10853 (1999).
16. Dressman, M. A. et al. Gene expression profiling detects gene amplification and differentiates tumor types in breast cancer. Cancer Res 63, 2194-2199 (2003).
17. Perera, R. J. et al. Identification of novel PPARgamma target genes in primary human adipocytes. Gene 369, 90-99 (2006).
18. Fontaine, C. et al. The orphan nuclear receptor Rev-Erbalpha is a peroxisome proliferator-activated receptor (PPAR) gamma target gene and promotes PPARgamma-induced adipocyte differentiation. J Biol Chem 278, 37672-37680 (2003).
19. Yin, L. et al. Rev-erbalpha, a heme sensor that coordinates metabolic and circadian pathways. Science 318, 1786-1789 (2007).
20. Yin, L. & Lazar, M. A. The orphan nuclear receptor Rev-erbalpha recruits the NCoR/histone deacetylase 3 corepressor to regulate the circadian Bmall gene. Mol Endocrinol 19, 1452-1459 (2005).
21. Wang, J., Yin, L. & Lazar, M. A. The orphan nuclear receptor Rev-erb alpha regulates circadian expression of plasminogen activator inhibitor type 1. J Biol Chem 281, 33842-33848 (2006).
22. Menendez, J. A. & Lupu, R. Fatty acid synthase and the lipogenic phenotype in cancer pathogenesis. Nat Rev Cancer 7, 763-777 (2007).
23. Kuhajda, F. P. Fatty acid synthase and cancer: new application of an old pathway. Cancer Res 66, 5977-5980 (2006).
24. Hatzivassiliou, G. et al. ATP citrate lyase inhibition can suppress tumor cell growth. Cancer Cell 8, 311-321 (2005).
25. DeBerardinis, R. J., Lum, J. J., Hatzivassiliou, G. & Thompson, C. B. The biology of cancer: metabolic reprogramming fuels cell growth and proliferation. Cell Metab 7, 11-20 (2008).
26. Menendez, J. A., Colomer, R. & Lupu, R. Why does tumor-associated fatty acid synthase (oncogenic antigen-519) ignore dietary fatty acids? Med Hypotheses 64, 342-349 (2005).
27. Warburg, O., Posener, K., Negelein, E. Uber den stoffwechsel der tumoren. Biochem Z 152, 319-344 (1924).
28. Rupert, B. E., Segar, J. L., Schutte, B. C. & Scholz, T. D. Metabolic adaptation of the hypertrophied heart: role of the malate/aspartate and alpha-glycerophosphate shuttles. J Mol Cell Cardiol 32, 2287-2297 (2000).
29. Guay, C., Madiraju, S. R., Aumais, A., Joly, E. & Prentki, M. A role for ATP-citrate lyase, malic enzyme, and pyruvate/citrate cycling in glucose-induced insulin secretion. J Biol Chem 282, 35657-35665 (2007).
30. Filipski, E. et al. Effects of light and food schedules on liver and tumor molecular clocks in mice. J Natl Cancer Inst 97, 507-517 (2005).
31. Silva, J. M. et al. Second-generation shRNA libraries covering the mouse and human genomes. Nat Genet 37, 1281-1288 (2005).
32. Folch, J., Lees, M. & Sloane Stanley, G. H. A simple method for the isolation and purification of total lipids from animal tissues. J Biol Chem 226, 497-509 (1957).

TABLE S1
Several shRNA sequences used in the present
study*

| shRNA | mature sequence |
|---|---|
| ERBB2 | CCCTGGCCGTGCTAGACAA |
| NR1D1 | GGCATGGTGTTACTGTGTA |
| PBP | CCGAGTTCCTCTTATCCTA |
| MAP2K6 | CAGATGACCTGGAGCCTAT |
| SPINT1 | CTGTGTAGTTTGTGCTGTA |
| PFN2 | AGCATTACGCCAATAGAAA |
| MKI67 | GCTACAAACTCCTAAGGAA |
| TPD52 | CTGTGAGATTCCTACCTTT |
| BNIP3L | AGCAGCAATGGCAATGATA |
| H2AFY | AAGTTTGTGATCCACTGTA |
| LASP1 | GGACCAGATCAGTAATATA |
| SIAT4C | ATTATTTAATGGGCTATTT |
| SFRS7 | GCTAGTATGTTGGAAGTTA |
| CA9 | CTTTGAATGGGCGAGTGAT |
| RPL19 | GCAAGAAGAAGGTCTGGTT |
| FADS2 | CCCATAGGGAGCTGATCGT |
| ERBB3 | CTACCAGTTGGAACACTTA |
| GRB7 | CTCGCCATCTGCATCCATC |
| STARD3 | AGGAGATCATCCAGTACAA |
| FASN | CTGGCCCAGGCTGAAGTTT |
| ACLY | GGGAGGAAGCTGATGAATA |
| ACACA | CACATGACCTTAAGATTAT |
| MDH1 | CGAGCTAAAGCTCAAATTG |
| ME1 | GGCTTTATCCTCCTTTGAA |

*all shRNA sequences used can be retrieved in http://codex.cshl.edu

TABLE S2
The primer pairs used for qRT-PCR

| | | |
|---|---|---|
| gene | forward primer | reverse primer |

| | | |
|---|---|---|
| ERBB2 | 5′-AGACACGTTTGAGTCCATGCC-3′ | 5′-ATCCCACGTCCGTAGAAAGGT-3′ |
| NR1D1 | 5′-TTCTTCCTCATCTTCCTCGTCG-3′ | 5′-CGTCCCCACACACTTTACACAG-3′ |
| PBP | 5′-GGCAACAACCCAATGAGTGGT-3′ | 5′-ATGCCGATCTTTGATGCTCATG-3′ |
| FASN | 5′-CCGTGGACCTGATCATCAAGAG-3′ | 5′-TCGATGACGTGGACGGATACT-3′ |
| ACLY | 5′-AAGATCTCGTGGCCAATGGA-3′ | 5′-AGGTTTGCGGATCAAACCAA-3′ |
| ACACA | 5′-CTTTGTGCCCACGGTTATCA-3′ | 5′-AGTGGTCCCTGTTTGTCTCCA-3′ |
| MDHI | 5′-TGCAAGGAAAGGAAGTTGGTG-3′ | 5′-TTCGAGCCTTGATGACAGCAG-3′ |
| FADS2 | 5′TGGTCATTGACCGCAAGGTT-3′ | 5′-AGGCATCCGTTGCATCTTCTC-3′ |
| ME1 | 5′-GCCATTGTGGTGACTGATGGA-3′ | 5′-TCATCCCTCCGCAAGCTGTAT-3′ |
| aP2 | 5′-GCATGGCCAAACCTAACATGAT-3′ | 5′-CCTGGCCCAGTATGAAGGAAA-3′ |
| β-actin | 5′-CTGTCCACCTTCCAGCAGATGT-3′ | 5′-CGCAACTAAGTCATAGTCCGCC-3′ |

1. A method of treating cancer, comprising:
a) providing a subject with breast cancer cells and an inhibitor of a gene encoding a protein associated with adipogenesis;
b) treating said subject with said inhibitor.

2. The method of claim 1, wherein said protein is a transcriptional regulator of fat synthesis and storage.

3. The method of claim 3, wherein said protein is NR1D1 (RevErb-alpha).

4. The method of claim 3, wherein said protein is PBP.

5. The method of claim 1, wherein said inhibitor comprises a short hairpin RNA selected from the group consisting of SEQ ID NO:1-2.

6. The method of claim 5, wherein treating with said RNA results in reduced proliferation of said breast cancer cells.

7. A method of treating cancer, comprising:
a) providing a subject with breast cancer cells and an inhibitor of a gene encoding a protein associated with lipid metabolism:
b) treating said subject with said inhibitor.

8. The method of claim 7, wherein said protein associated with adipogenesis is selected from a fatty acid synthase and a fatty acid desaturase.

9. The method of claim 7, wherein said inhibitor comprises a short hairpin RNA selected from the group consisting of SEQ ID NO: 3-4.

10. The method of claim 9, wherein treating with said RNA results in reduced proliferation of said breast cancer cells.

11. A method of identifying a test agent that affects the expression of a transcription factor selected from the group consisting of NR1D1, PBP, and PPARγ, the method comprising: (i) providing a first and a second cell expressing the transcription factor, (ii) contacting the first cell with the test agent, (iii) contacting the second cell with a control agent, (iii) measuring expression of the transcription factor in the first and second cells, and (iv) comparing the amount of expression of the transcription factor in the first and second cells to determine whether or not the test agent promotes or inhibits the expression of the transcription factor.

12. A method of identifying a test agent that affects the expression of a protein translated from the message transcribed under the influence of the transcription factor, the method comprising: (i) providing a first and a second cell expressing the transcription factor, (ii) contacting the first cell with the test agent, (iii) contacting the second cell with control agent, (iii) measuring expression of the protein in the first and second cells, and (iv) comparing the amount of expression of the protein in the first and second cells to determine whether or not the test agent promotes or inhibits the expression of the protein.

13. A method of identifying a test agent that affects the activity of a protein translated from the message transcribed under the influence of the transcription factor, the method comprising: (i) providing a first and a second amount of the protein, (ii) contacting the first and second amounts with a substrate of the protein under conditions in which a product forms, expressing the transcription factor, (ii) contacting the first protein amount with the test agent, (iii) contacting the second protein amount with a control agent, (iii) measuring a first rate of formation of the product by the first protein amount and a second rate of production the second protein amount, and (iv) comparing the first and second rates to determine whether or not the test agent promotes or inhibits the activity of the protein.

14. The method of claim 13 wherein the amounts are in vitro.

15. The method of claim 13 wherein the amounts are in cell-free systems.

16. The method of laiml3 wherein the amounts are in cells.

17. A method of identifying a test agent that affects the proliferation of a cancer cell, the method comprising: (i) providing a first and a second cancer cell, (ii) contacting the first cell with the test agent, (iii) contacting the second cell with a control agent, (iii) measuring a first rate of proliferation of said first cell and a second rate of proliferation of said second cell, and (iv) comparing the first and second rates to determine whether or not the test agent promotes or inhibits the rate of proliferation of the cancer cell.
